(19) United States
(12) Patent Application Publication
(10) Pub. No.: US 2022/0218051 A1
(43) Pub. Date: Jul. 14, 2022

(54) FACE SHIELD DEVICE FOR SMART APPLICATIONS

(71) Applicant: Ahmad SALEH, Dubai (AE)

(72) Inventor: Ahmad SALEH, Dubai (AE)

(21) Appl. No.: 17/535,817

(22) Filed: Nov. 26, 2021

Related U.S. Application Data
(60) Provisional application No. 63/118,643, filed on Nov. 26, 2020.

(51) Int. Cl. A41D 13/11; A61B 5/01; G01K 3/00

(52) U.S. Cl. CPC A41D 13/1184; G01K 13/20; G01K 3/005; A61B 5/01

(57) ABSTRACT

A portable face shield device (100) for preventing or reducing the spread of infectious diseases designed to integrate an electronic circuitry for smart applications, and a process (200) for manufacturing the portable face shield (100) are disclosed. The face shield (100) comprises a frame (102) for supporting the face shield device (100) on user's head and at least one shell (106) coupled to the frame (102) for housing an electronic circuitry (104). The process (200) for manufacturing the face shield device (100) includes providing (202) the frame (102) to be worn around the head of a user and connecting (206) the at least one shell (106) on the frame (102) such that the shell (106) house the electronic circuitry (104).

### CROSS-REFERENCE TO RELATED APPLICATIONS AND PRIORITY

This patent application claims priority from U.S. Provisional Patent Application No. 63/118,643 filed Nov. 26, 2020. This patent application is herein incorporated by reference in its entirety.

### FIELD OF THE INVENTION

The present invention relates to a personal protective equipment, and more particularly, to a face shield device adapted to integrate an electronic circuitry for smart applications, for wear by a user in order to prevent or reduce the spread of infectious diseases including but not limited to COVID-19 all in enabling embedded smart applications.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Hundreds of million of students from prekindergarten through primary and secondary grades globally have had their education disrupted by school closures amid the continuing spread of a novel coronavirus COVID-19, a figure the U.N. agency dealing with the crisis says is unprecedented. There are school closures of some kind in dozens of countries on three continents with hundreds of millions of students around the world facing upheaval, including many countries that have shut schools nationwide. The United Nations has warned of the unparalleled scale and speed of the educational disruption being caused by coronavirus.

Few schools have closed, and others are considering doing that, if necessary. They have also started taking precautions to prevent virus outbreaks, including canceling trips, teaching students to wash their hands for an appropriate period of time with soapy water and sending reassuring messages to parents.

Schools have implemented the wearing of face shields or masks as mandatory precautionary measures. Face shields are simple, transparent screens that cover the face and help prevent infectious droplets from entering the eyes, nose and mouth. They are usually worn for blocking splashes and sprays from reaching the face and making it preventing users from touching their faces.

Schools, hospitals, and other institutions have been facing increasing challenging of controlling and managing the wear of masks and face shields within their institutions, in addition to the challenges for taking precautionary measures as well as corrective measures for enhancing the safety and helath conditions of their members. Though these measures have been proved to be one of the most implemented and recommended preventive measures among others, there is a lot of difficulty also associated with their usage. In fact, face shield is often preferred over face masks for several reasons. Firstly, face shields cover the whole face from head extending all the way below the chin, instead of just the mouth and nose area (as in the case of face masks). As a result, the face shield prevents the user from touching his/her own face and eyes that may trigger spreading of the virus. It has been observed in a recently conducted study that face shields reduce the inhalation exposure of the exposure of the user by 96 percent within 18 inches of a cough.

A further advantage associated with the use of face shields is that they are comfortable and convenient to be worm by the user and being made of transparent material, allow other to see the user's expressions or read their lips, pavin for better communication without the need to take off the shield.

The conventional face shields as described above, do have a number of advantages, but there is still room for improvement. Also, traditional face shields do not integrate or enable smart functions and features.

It is, therefore, an object of the invention to provide an improved portable face shield which offers additional functionality to the users and enhances the performance of the conventional face shields while offering ease to wear with comfort and stability during prolonged usage of the face shield by the user.

### SUMMARY OF THE INVENTION

This summary is provided to introduce a selection of concepts in a simplified format that are further described in the detailed description of the present disclosure. This summary is not intended to identify key or essential inventive concepts of the present disclosure, nor is it intended for determining the scope of the present disclosure.

According to an embodiment of the present disclosure, a face shield device comprises a frame for supporting the face shield device on user's head, and at least one shell coupled to the frame for housing the electronic circuitry.

According to an embodiment of the present disclosure, the face shield device comprises at least one electronic circuitry comprising a plurality of electronic components for the operation of the device.

According to an embodiment of the present disclosure, wherein the frame may be a belt.

According to an embodiment of the present disclosure, the frame is U-shaped or semi-circular in shape.

According to an embodiment of the present disclosure, the frame is symmetric about a centre axis and variable such as thickness is constant around the circumference.

According to an embodiment of the present disclosure, the frame is made of silicone.

According to an embodiment of the present disclosure, the frame is fixed to a pad.

According to an embodiment of the present disclosure, wherein the pad is made of polifoam.

According to an embodiment of the present disclosure, the at least one shell are two shells.

According to an embodiment of the present disclosure, the two shells are positioned opposite each other on each side of the frame such that the two circuitry housings are symmetrical with respect to the center axis of the frame.

According to an embodiment of the present disclosure, the weight of the shell is divided equally between the two shells such that the weight of the circuitry is distributed equally on both sides of the face shield device.

According to an embodiment of the present disclosure, the at least one shell is made of polyethylene terephthalate glycol (PETG).

According to an embodiment of the present disclosure, the at least one of the shell comprises a user interface for enabling the user to send a wireless signal.

According to an embodiment of the present disclosure, the user interface comprises a button.

According to an embodiment of the present disclosure, the button is a switching button.

According to an embodiment of the present disclosure, the wireless signal is an access control signal.

According to an embodiment of the present disclosure, the face shield device further comprising a USB port.

According to an embodiment of the present disclosure, the shell comprises one or more buttons for operating the electronic circuit components.

According to an embodiment of the present disclosure, the at least one shell comprises a door for providing access to the plurality of electronic circuit components.

According to an embodiment of the present disclosure, the at least one shell is located above the ear of the user for enabling the user to access the user interface while wearing the device.

According to an embodiment of the present disclosure, the portable face shield device comprises a shield connected to the frame.

According to an embodiment of the present disclosure, the shield is made of polyethylene terephthalate glycol (PETG) or other transparent material.

According to an embodiment of the present disclosure, the shield is removably connected to the frame.

According to an embodiment of the present disclosure, the portable face shield comprises a fastening unit.

According to an embodiment of the present disclosure, the ends of the fastening unit are connected to ends of the frame each.

According to an embodiment of the present disclosure, the fastening unit is made of an elastic material.

According to an embodiment of the present disclosure, the elastic material is rubber.

According to an embodiment of the present disclosure, the shield comprises a lining on one of the sides adapted to be connected to the portable face shield device.

According to an embodiment of the present disclosure, the lining is connected to the device at one or more connection points on the outer surface of the shell.

According to an embodiment of the present disclosure, the connection point is a bolted joint configured to be rotated against the frame.

According to an embodiment of the present disclosure, the one or more rotatable connection points are configured to adjust the lining of the shield to at least three positions (P1, P2, P3) longitudinally along the face of the user.

According to an embodiment of the present disclosure, the fastening unit is integrated with the frame.

According to an embodiment of the present disclosure, the frame comprises a rotatable band configured to be rotated along the head of the user; and a locking mechanism for controlling the rotation of the rotatable band.

According to an embodiment of the present disclosure, the ends of the rotatable band are fixed to the inner surface of the shell at one or more joints.

According to an embodiment of the present disclosure, the joints are configured to be rotated.

According to an embodiment of the present disclosure, the locking mechanism) comprises a curve on the inner surface of the shell allowing the rotating of the rotatable band along the boundaries of the curve.

According to an embodiment of the present disclosure, the locking mechanism comprises a sliding lock at the junction connecting the end of the rotatable band and an end of a connecting unit on the inner surface of the shell.

According to an embodiment of the present disclosure, the locking mechanism is configured to adjust the rotatable band to at least two positions (F1, F2).

According to an embodiment of the present disclosure, at one (F1) of the at least two positions (F1, F2), the rotatable band is on the forehead of the user.

According to an embodiment of the present disclosure, at one (F2) of the at least two positions (F1, F2), the rotatable band is on the head of the user.

According to an embodiment of the present disclosure, a process of manufacturing a face shield device comprising a frame, and at least one shell coupled to the frame for housing the electronic circuitry comprises providing the frame to be worn around the head of a user and connecting the at least one shell on the frame such that the shell house the electronic circuitry.

According to an embodiment of the present disclosure, the frame is bent and laser cut from a bendable sheet material to form a semi-circular shaped configuration.

According to an embodiment of the present disclosure, further comprising fixing a pad to the frame.

To further clarify the advantages and features of the present disclosure, a more particular description of the disclosure will be rendered by reference to specific embodiments thereof, which is illustrated in the appended drawings. It is appreciated that these drawings depict only typical embodiments of the disclosure and are therefore not to be considered limiting of its scope. The disclosure will be described and explained with additional specificity and detail with the accompanying drawings.

### BRIEF DESCRIPTION OF THE DRAWINGS

The subject matter that is regarded as the invention is particularly pointed out and distinctly claimed in the claims at the conclusion of the specification. The foregoing and other aspects, features, and advantages of the invention are apparent from the following detailed description taken in conjunction with the accompanying drawings in which:

FIG. 1 illustrates an overall schematic view of a portable face shield for smart applications, in accordance with an embodiment of the present disclosure;

FIGS. 2a and 2b illustrate a perspective view of the portable face shield for smart applications, in accordance with an embodiment of the present disclosure;

FIGS. 2c and 2d illustrate a left side view of the portable face shield for smart applications, in accordance with an embodiment of the present disclosure;

FIG. 2e illustrates a front view of the portable face shield, in accordance with an embodiment of the present disclosure;

FIG. 2f illustrates a front top view of the portable face shield, in accordance with an embodiment of the present disclosure;

FIG. 2g illustrates a back view of the portable face shield, in accordance with an embodiment of the present disclosure;

FIG. 2h illustrates a back top view of the portable face shield, in accordance with an embodiment of the present disclosure;

FIG. 2i illustrates a right side view of the portable face shield, in accordance with an embodiment of the present disclosure;

FIGS. 3a and 3b illustrate different close up views of the portable face shield, in accordance with an embodiment of the present disclosure;

FIG. 4a, FIG. 4b, FIG. 4c, FIG. 4d, FIG. 4e, FIG. 4f, FIG. 4g, FIG. 4h, FIG. 4i, FIG. 4j, FIG. 4k, and FIG. 4l illustrate different views of the portable face shield device 100 depicting rotating features according to an embodiment of the present disclosure.

FIGS. 4a and 4b illustrate a perspective view of the portable face shield, in accordance with an embodiment of the present disclosure.

FIGS. 4c and 4d illustrate a left side view of the portable face shield in three different head positions, in accordance with an embodiment of the present disclosure.

FIGS. 4e and 4f illustrate a right side view of the portable face shield in three different head positions, in accordance with an embodiment of the present disclosure.

FIGS. 4g and 4h illustrate a front view of the portable face shield in three different head positions, in accordance with an embodiment of the present disclosure.

FIGS. 4i and 4j illustrate a back view of the portable face shield, in accordance with an embodiment of the present disclosure.

FIGS. 4k and 4l illustrates a back top view of the portable face shield, in accordance with an embodiment of the present disclosure.

FIGS. 5a and 5b illustrate an exploded view of the portable face shield, in accordance with an embodiment of the present disclosure.

FIGS. 6a and 6b illustrate a perspective view of the portable face shield, in accordance with an embodiment of the present disclosure.

FIG. 6c illustrates a left and right side view of the portable face shield, in accordance with an embodiment of the present disclosure.

FIG. 6d illustrate a front and back side view of the portable face shield, in accordance with an embodiment of the present disclosure.

FIG. 6e illustrate back side view of the portable face shield, in accordance with an embodiment of the present disclosure.

FIGS. 7a, 7b, 7c, 7d, 7e and 7f illustrate different views of the portable face shield in two different frame positions, in accordance with an embodiment of the present disclosure.

FIGS. 7g and 7h illustrate a top view of the portable face shield in two different frame positions, in accordance with an embodiment of the present disclosure.

FIG. 7i illustrates a right side view of the portable face shield in two different frame positions, in accordance with an embodiment of the present disclosure.

FIG. 7j illustrates a left side view of the portable face shield in two different frame positions, in accordance with an embodiment of the present disclosure.

FIG. 7k illustrates a back side view of the portable face shield in two different frame positions, in accordance with an embodiment of the present disclosure.

FIG. 7l illustrates a front side view of the portable face shield in two different frame positions, in accordance with an embodiment of the present disclosure.

FIGS. 8a, 8b, 9a and 9b illustrate close up views of the connection points in the portable face shield in two different frame positions, in accordance with an embodiment of the present disclosure.

FIGS. 10a to 10i, 11a to 11b, and 12a to 12c illustrate different exploded views of the portable face shield, in accordance with an embodiment of the present disclosure.

FIG. 13 illustrate left side X-ray view of electronic circuit components placement in a portable face shield, in accordance with an embodiment of the present disclosure,

FIG. 14 illustrates right side X-ray view of electronic circuit components placement in a portable face shield, in accordance with an embodiment of the present disclosure.

FIGS. 15a-15c illustrate different views of electronic circuit components placement in a portable face shield, in accordance with an embodiment of the present disclosure.

FIG. 16 illustrates a process of manufacturing the portable face shield, in accordance with an embodiment of the present disclosure.

Further, skilled artisans will appreciate that elements in the drawings are illustrated for simplicity and may not have necessarily been drawn to scale. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

### DETAILED DESCRIPTION OF THE INVENTION

For the purpose of promoting an understanding of the principles of the invention, reference will now be made to the embodiment illustrated in the drawings, and specific language will be used to describe the same. It will nevertheless be understood that no limitation of the scope of the invention is thereby intended. Such alterations and further modifications in the illustrated system, and such further applications of the principles of the invention as illustrated therein would be contemplated as would normally occur to one skilled in the art to which the invention relates. Unless otherwise defined, all technical and scientific terms used herein have the same meaning as commonly understood by one of ordinary skilled in the art. The system, methods, and examples provided herein are illustrative only and are not intended to be limiting.

The term “some” as used herein is to be understood as “none or one or more than one or all.” Accordingly, the terms “none,” “one,” “more than one,” “more than one, but not all” or “all” would all fall under the definition of “some.” The term “some embodiments” may refer to no embodiments or to one embodiment or to several embodiments or to all embodiments, without departing from the scope of the present disclosure.

The terminology and structure employed herein is for describing, teaching, and illuminating some embodiments and their specific features. It does not in any way limit, restrict or reduce the spirit and scope of the claims or their equivalents.

More specifically, any terms used herein such as but not limited to “includes,” “comprises,” “has,” “consists,” and grammatical variants thereof do not specify an exact limitation or restriction and certainly do not exclude the possible addition of one or more features or elements, unless otherwise stated, and furthermore must not be taken to exclude the possible removal of one or more of the listed features and elements, unless otherwise stated with the limiting language “must comprise” or “needs to include.”

Whether or not a certain feature or element was limited to being used only once, either way, it may still be referred to as “one or more features” or “one or more elements” or “at least one feature” or “at least one element.” Furthermore, the use of the terms “one or more” or “at least one” feature or element do not preclude there being none of that feature or element, unless otherwise specified by limiting language such as “there needs to be one or more . . . ” or “one or more element is required.”

Unless otherwise defined, all terms, and especially any technical and/or scientific terms, used herein may be taken to have the same meaning as commonly understood by one having ordinary skills in the art.

Reference is made herein to some “embodiments.” It should be understood that an embodiment is an example of a possible implementation of any features and/or elements presented in the attached claims. Some embodiments have been described for the purpose of illuminating one or more of the potential ways in which the specific features and/or elements of the attached claims fulfill the requirements of uniqueness, utility and non-obviousness.

Use of the phrases and/or terms including, but not limited to, “a first embodiment,” “a further embodiment,” “an alternate embodiment,” “one embodiment,” “an embodiment,” “multiple embodiments,” “some embodiments,” “other embodiments,” “further embodiment”, “furthermore embodiment”, “additional embodiment” or variants thereof do not necessarily refer to the same embodiments. Unless otherwise specified, one or more particular features and/or elements described in connection with one or more embodiments may be found in one embodiment, or may be found in more than one embodiment, or may be found in all embodiments, or may be found in no embodiments. Although one or more features and/or elements may be described herein in the context of only a single embodiment, or alternatively in the context of more than one embodiment, or further alternatively in the context of all embodiments, the features and/or elements may instead be provided separately or in any appropriate combination or not at all. Conversely, any features and/or elements described in the context of separate embodiments may alternatively be realized as existing together in the context of a single embodiment.

Any particular and all details set forth herein are used in the context of some embodiments and therefore should not be necessarily taken as limiting factors to the attached claims. The attached claims and their legal equivalents can be realized in the context of embodiments other than the ones used as illustrative examples in the description below.

Embodiments of the present invention will be described below in detail with reference to the accompanying drawings.

The present disclosure provides a portable face shield device for smart applications (also called herein portable face shield or portable face shield device) 100. A user may wear the face shield device 100 (hereinafter “device”) for safety and security purposes, for example to prevent or reduce the spread of infectious diseases. The infectious diseases (interchangeably used as “virus”) may include but is not limited to the novel COVID19 corona virus.

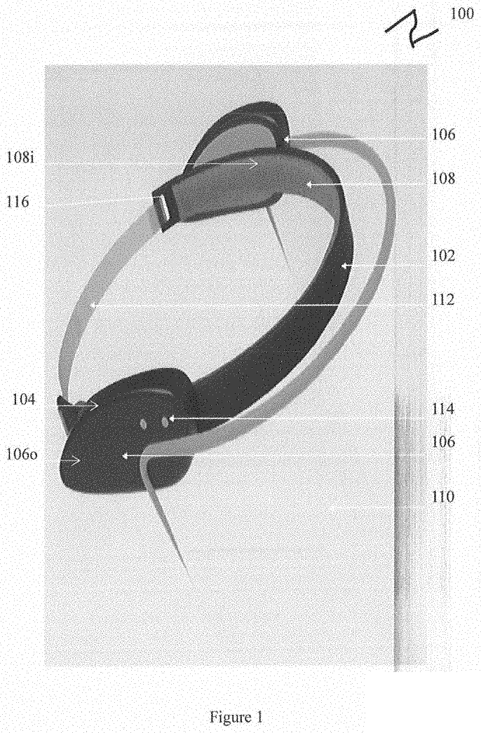

FIG. 1 illustrates an overall schematic view of a portable face shield 100 in accordance with an embodiment of the present disclosure. The portable face shield 100 may comprise a frame 102, and at least one shell 106 coupled to the frame 102. The frame 102 may be adapted for supporting the face shield device 100 on user's head and the at least one shell 106 may be adapted for housing an electronic circuitry 104. The portable face shield 100 may further comprise a fastening unit 112, preferably an elastic band to provide a grip on the user's head to hold the portable face shield 100 from behind, a face shield 110 and a pad 108.

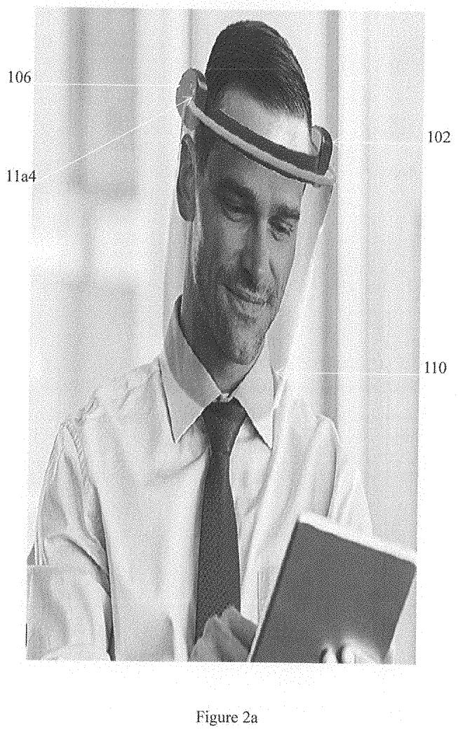

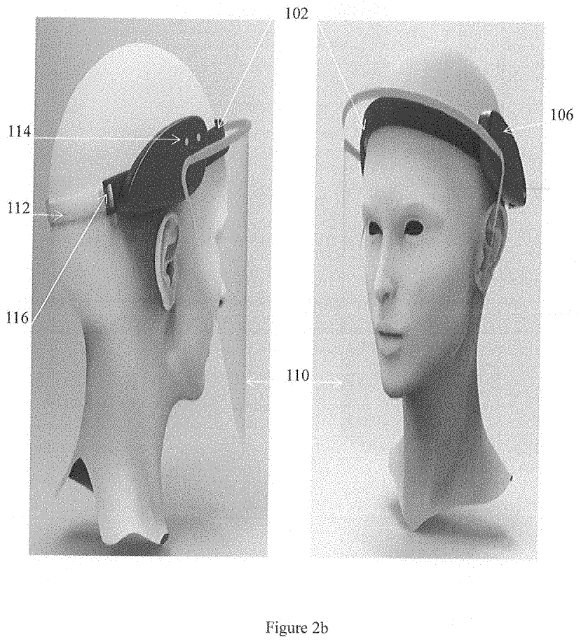

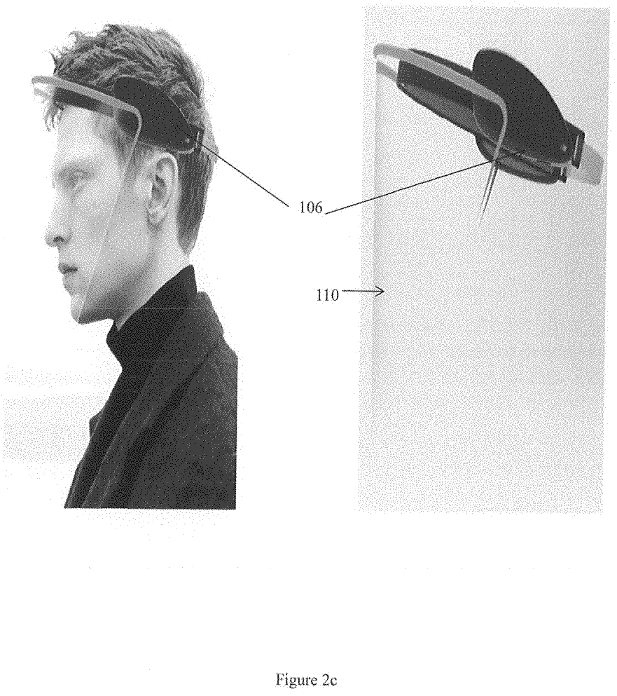

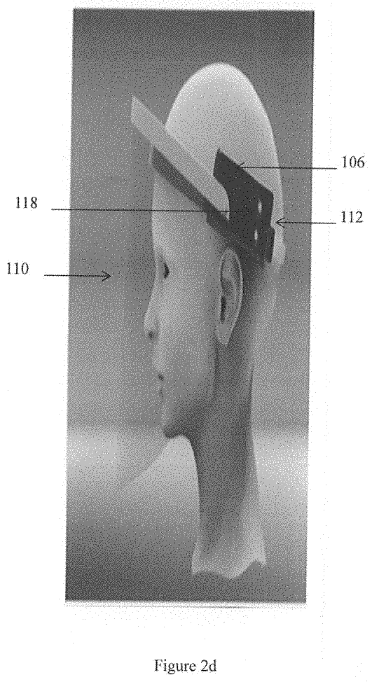

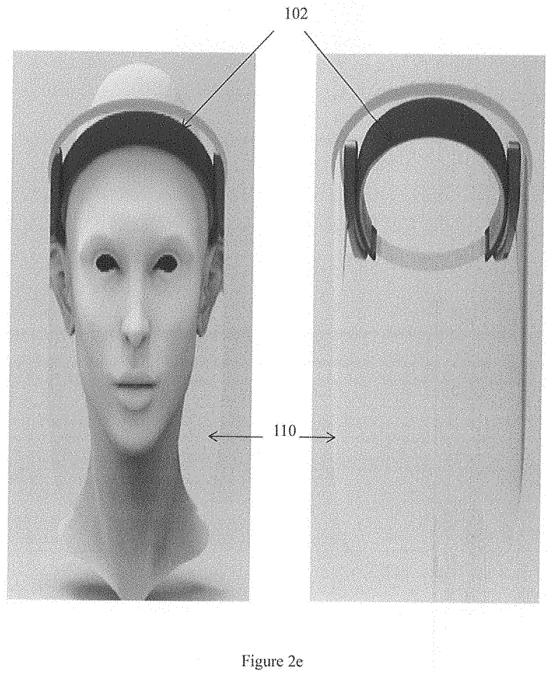

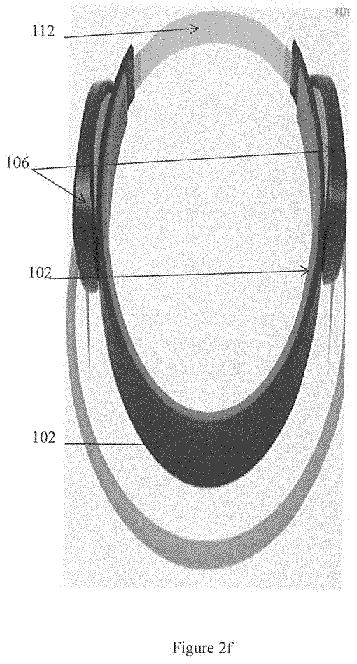

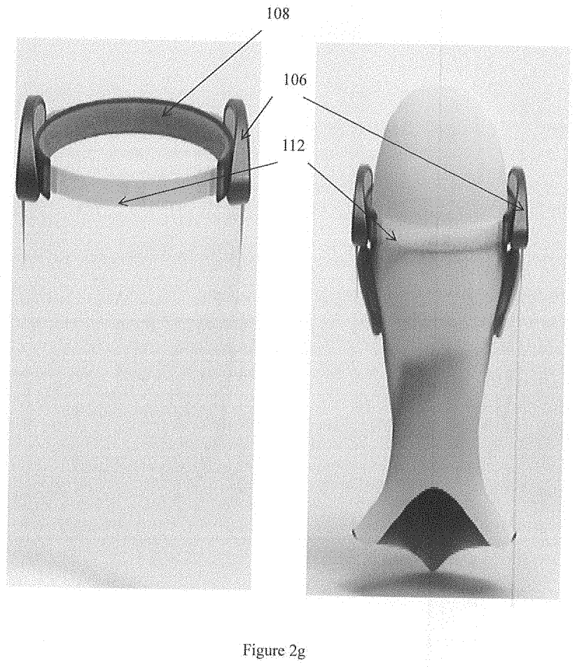

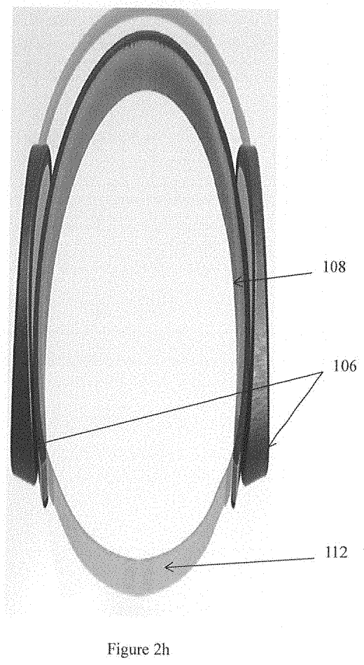

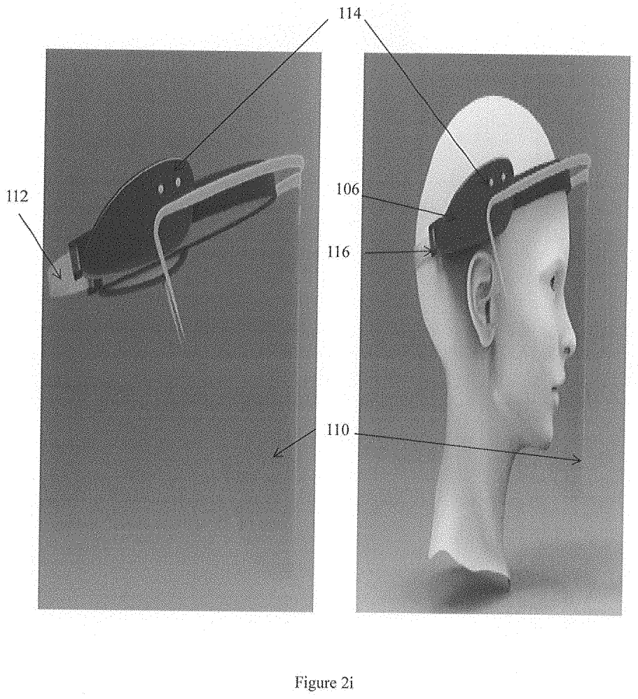

FIG. 2a, FIG. 2b, FIG. 2c, FIG. 2d, FIG. 2e, FIG. 2f, FIG. 2g, FIG. 2h, and FIG. 2i illustrate different views of the portable face shield device 100 according to an embodiment of the present disclosure. Particularly, FIGS. 2a and 2b illustrate a perspective view of the portable face shield, in accordance with an embodiment of the present disclosure. FIGS. 2c and 2d illustrate a left side view of the portable face shield, in accordance with an embodiment of the present disclosure. FIG. 2e illustrates a front view of the portable face shield, in accordance with an embodiment of the present disclosure. FIG. 2f illustrates a front top view of the portable face shield, in accordance with an embodiment of the present disclosure, FIG. 2g illustrates a back view of the portable face shield, in accordance with an embodiment of the present disclosure. FIG. 2h illustrates a back top view of the portable face shield, in accordance with an embodiment of the present disclosure, and FIG. 2i illustrates a right side view of the portable face shield, in accordance with an embodiment of the present disclosure.

In accordance to an embodiment of FIG. 1 and FIGS. 2a to 2i, the frame 102 may be made of a silicone belt for a simple design. Particularly, the frame 102 may be made of silicone to make it resistant to many chemicals, greases, and oils and therefore, easy to maintain and use. Additionally, the material of the frame 102 may offer exceptional anti-adhesive effect while offering stability to the device 100 on the forehead of the user. The frame 102 may be U-shaped or semi-circular in shape with at least one slot 116 at each of the ends of the frame 102. The frame 102 may be symmetric about a centre axis and variable such as thickness may be constant around the circumference. The frame 102 may comprise an outer surface 102o and an inner surface 102i wherein the inner surface 102i of the frame 102 may come in contact with the head of the user when worn by the user.

In an embodiment, the inner surface 102i of the frame 102 may house the pad 108. The pad 108 may comprise of an outer surface 108o fixed to the frame 102 in a way such that an outer surface 108o of the pad 108 is in contact with the inner surface 102i of the frame 102 and the inner surface 108i of the pad 108 is in contact with the head of the user when the user wears the device 100. The pad 108 may be made of polifoam or other semi rigid or rubbery foam composed of minute bubbles of air or carbon dioxide embedded in a polymer matrix, according to an embodiment of the present disclosure. The pad 108 may provide additional softness and support to the device 100, thus adding comfort even during prolonged usage of the device 100 by the user. Referring to FIG. 1, the device 100 may comprise a fastening unit 112 positioned at the back side of the device 100. Referring to FIGS. 2g and 2h, the back side of the device 100 may be opposite to the side that is placed on the head of the user. The fastening unit 112 may comprise an adjustable size fabric fastener, the two ends of which may be connected to the respective slots 116 on the ends of the frame 102.

In an embodiment, the fastening unit 112 may be made of an elastic material that may include but is not limited to a rubber band, such that it adjusts itself according to the head of the user when wore and stays stable while the user moves. In another embodiment, the fastener unit 1012 may have an adjusting mechanism comprising multiple predefined adjusting positions on the length of the fastener. The user may opt for any of the predefined positions based on his/her comfort while wearing the device 100.

Further, the device 100 may comprise a shield 110 positioned at front and connected to the shell 106. The shield 110 may be made of a transparent material including but not limited to polyethylene terephthalate glycol (PETG). The shield 110 made of PETG may also be painted, silk screened or hot stamped. The shield 110 may be cut in a rectangular shape and curved through the portion with shorter length so as to make an arc shaped structure. One of the portions with shorter length may be framed with silicone or other bendable material. The framed curved portion may then be attached to an outer surface 106o of the shell 106. The connection may be made at the two ends of the shield 110 and it may cover the face of the user. The shield 110 may be removable attached to the shell 106 such that it may be detached and replaced when damaged or when to be cleaned.

As shown in FIG. 2c, the at least one shell 106 may be a shell shaped structure configured to house the electronic circuitry. In an embodiment, two shells 106 may be positioned opposite each other on each side of the frame 102 such that the two shells 106 are symmetrical with respect to the center axis of the frame 102. The shell 106 may be fixed to the frame 102 in a way that the hollow portion of the shell 106 may face the outer surface 102o of the frame 102. The shell 106 may allow an access to a plurality of electronic circuit components in the device 100. The weight of the shell 106 is divided equally between the two shells 106 such that the weight of the circuitry 104 is distributed equally on both sides of the device 100. The shell 106 may be made of polyethylene terephthalate glycol (PETG), according to an embodiment.

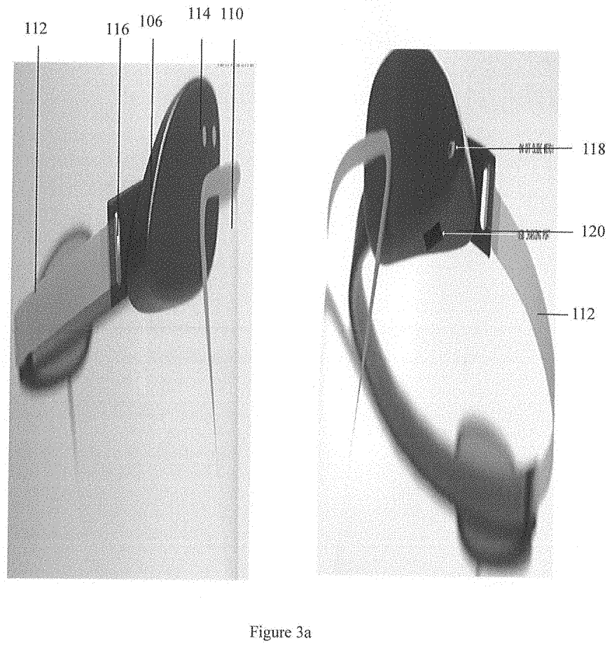

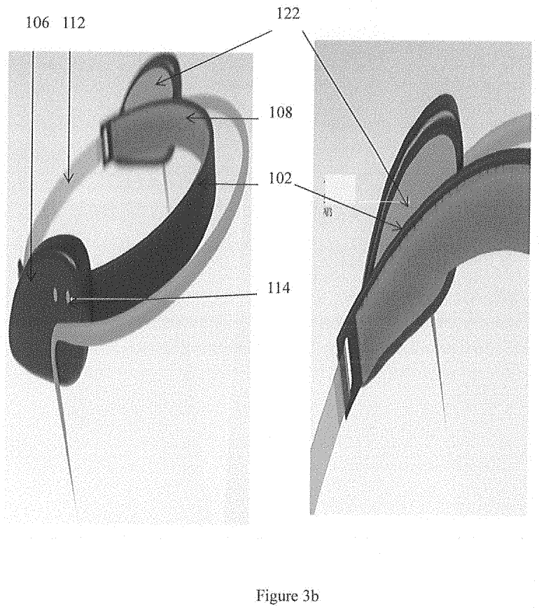

FIGS. 3a and 3b illustrate different close up views of the device 100, in accordance with an embodiment of the present disclosure. The location of the shell 106 on the frame 102 enables an easy access to the user. The shell 106 may comprise a user interface for enabling the user to send a wireless signal. The wireless signal may be an access control signal according to an embodiment. The user interface may comprise a button 118 at the outer surface 106o. The button 118 may be a switching button. The user may switch ON/OFF an electronic circuitry 104 placed inside the shell 106. In a further embodiment, the shell 106 may comprise one or more operating buttons 114 (interchangeably used as “buttons”) on the outer surface 106o of one of the shell 106 of the device 100. In an embodiment, said buttons 114 may be positioned on the portion of the shell 106 such that the user may easily operate the buttons 114 with his/her hands. The buttons 114 may be push buttons or slide buttons. In an embodiment, the buttons 112 may allow operating one or more electronic circuit components in the portable face shield 100.

The electronic circuitry 104 comprising a plurality of electronic circuit components for operating the switching button 118. The circuitry 104 may be put to OFF position when not in use or when the user intends not to the use electronic circuit components. Referring to FIG. 3a, in an embodiment, the shell 106 may further comprise a USB port 120 positioned on the outer surface of the shell 106o. The USB port 120 may allow charging of the device 100 on being connected to a host device. The host device may be a personal computer according to an embodiment of the invention. Additionally, the USB port 120 may enable the user to upload data to and/or download data from the host device. The user may be able to personalize the device 100 by himself setting the preset values for the electronic circuit components. This may enhance the user experience associated with the device 100.

Referring to FIG. 3b, in an embodiment, the shell 106 may comprise at least one door 122 on the inner surface 106i. The door 122 may enable access to the electronic circuitry 104 and thereby, the electronic circuit components in the device 100. In an embodiment, the door 122 may be openable by detaching a portion of the inner surface 106i of the shell 106 that may be detachably attached to the shell 106. This may facilitate easy maintenance of the device 100 by either repairing or replacing the electronic circuit components.

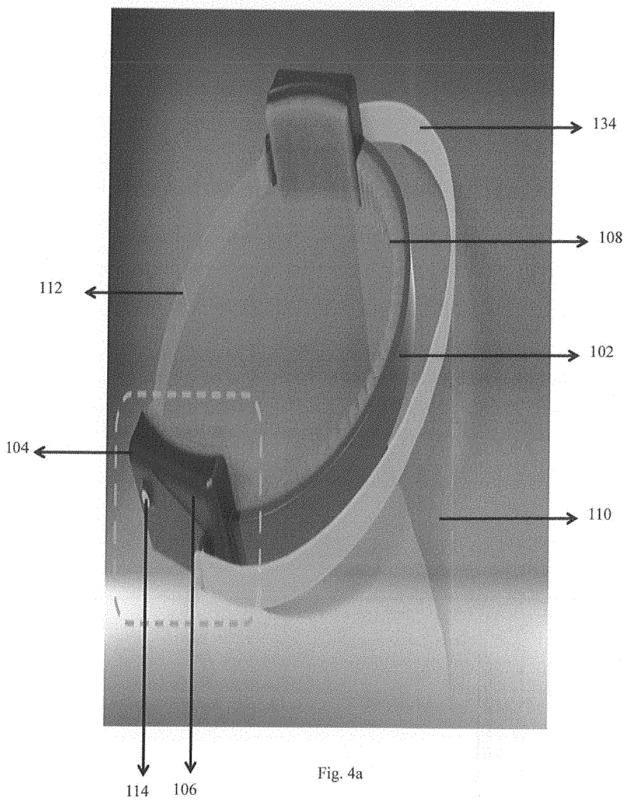

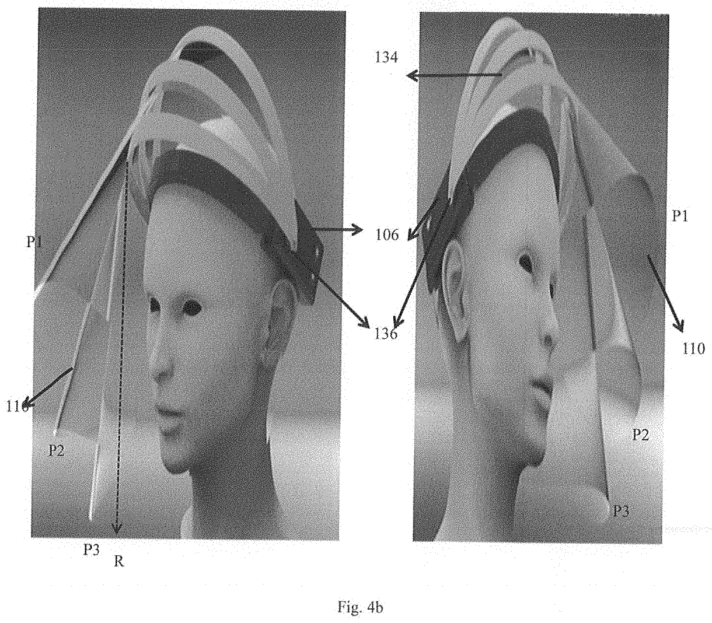

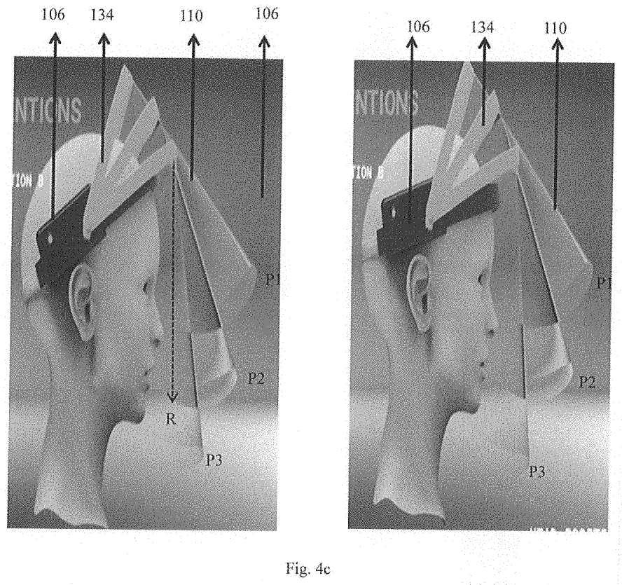

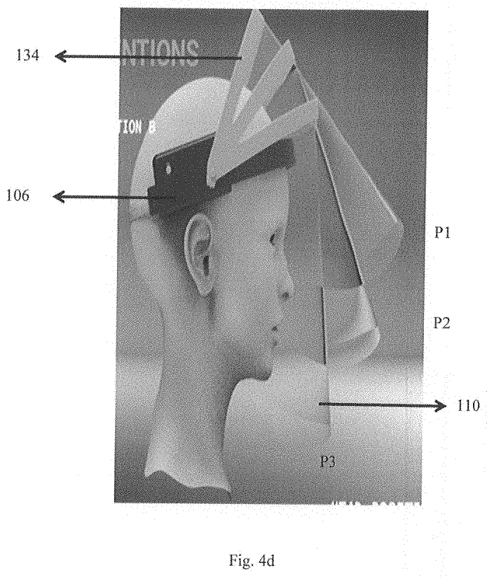

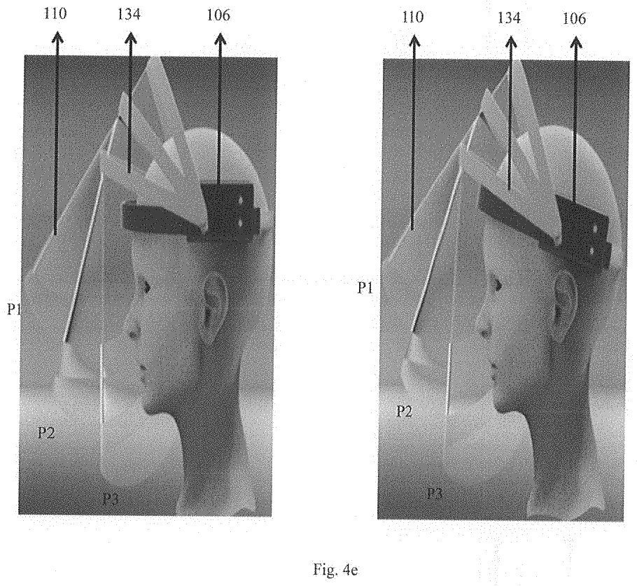

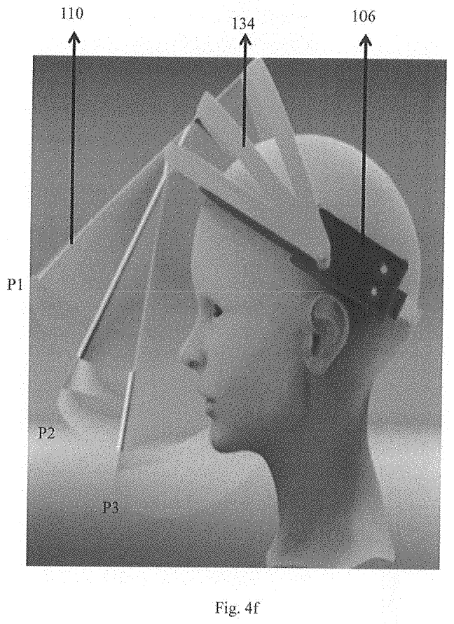

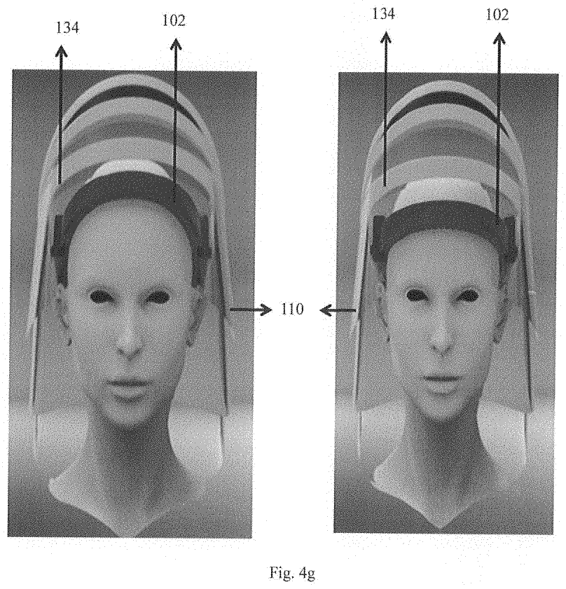

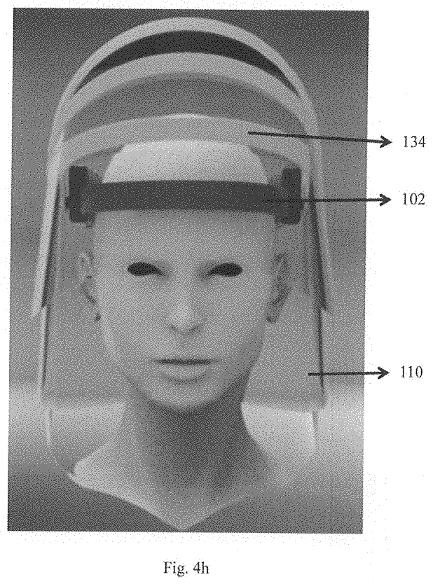

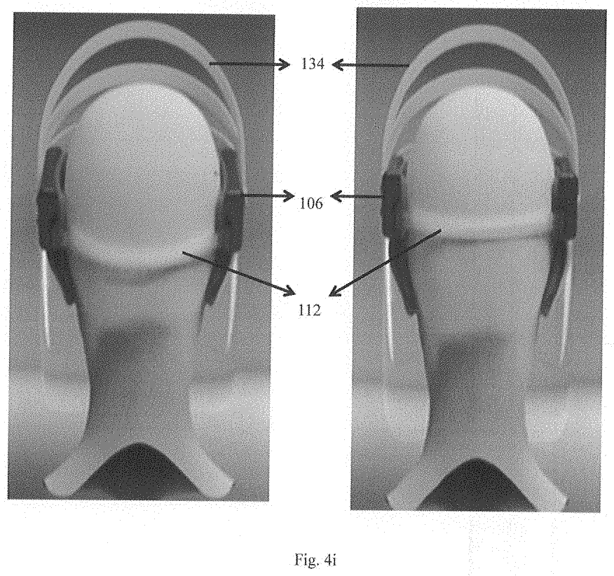

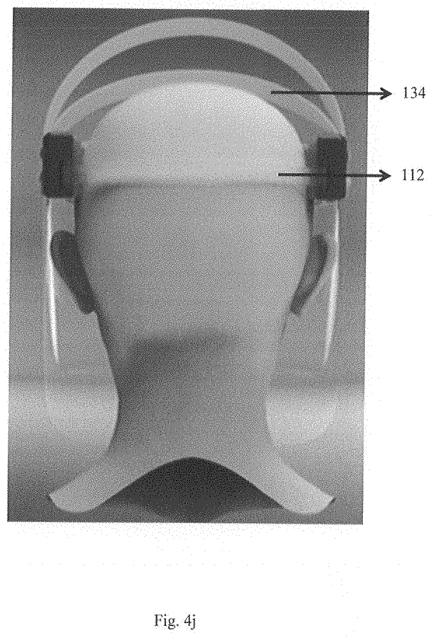

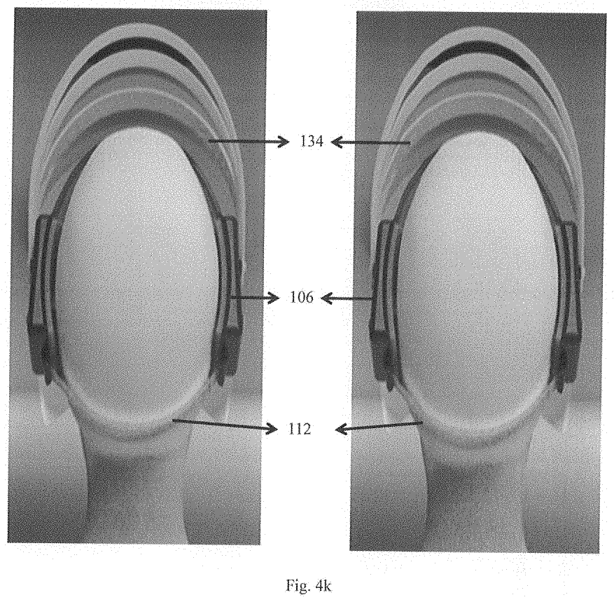

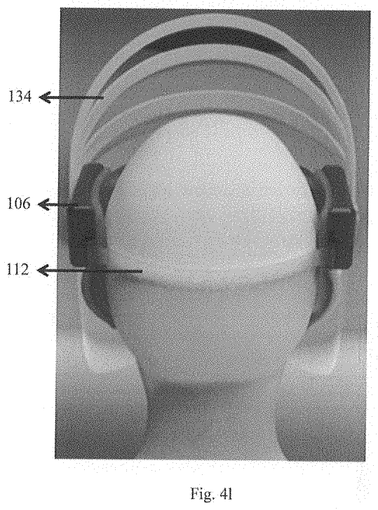

FIGS. 4a and 4b illustrate an overall schematic view of a portable face shield 100 in accordance with an embodiment of the present disclosure. The portable face shield 100 may comprise a frame 102, and at least one shell 106 coupled to the frame 102. The frame 102 may be adapted for supporting the face shield device 100 on user's head and the at least one shell 106 may be adapted for housing an electronic circuitry. The portable face shield 100 may further comprise of a fastening unit 112, preferably an elastic band to provide a grip on the user's head to hold the portable face shield 100 from behind, a rotatable face shield 110 (hereinafter referred to as “face shield”) and a pad 108. Further, FIGS. 4c-4l illustrate different view of the device 100. FIGS. 4c and 4d illustrate a left side view of the device 100 in three different head positions, in accordance with an embodiment of the present disclosure. FIGS. 4e and 4f illustrate a right side view of the device 100 in three different head positions, in accordance with an embodiment of the present disclosure. FIGS. 4g and 4h illustrate a front view of the device 100 in three different head positions, in accordance with an embodiment of the present disclosure. FIGS. 4i and 4j illustrate a back view of the device 100 and FIGS. 4k and 4l illustrate a back top view of the device 100, in accordance with an embodiment of the present disclosure.

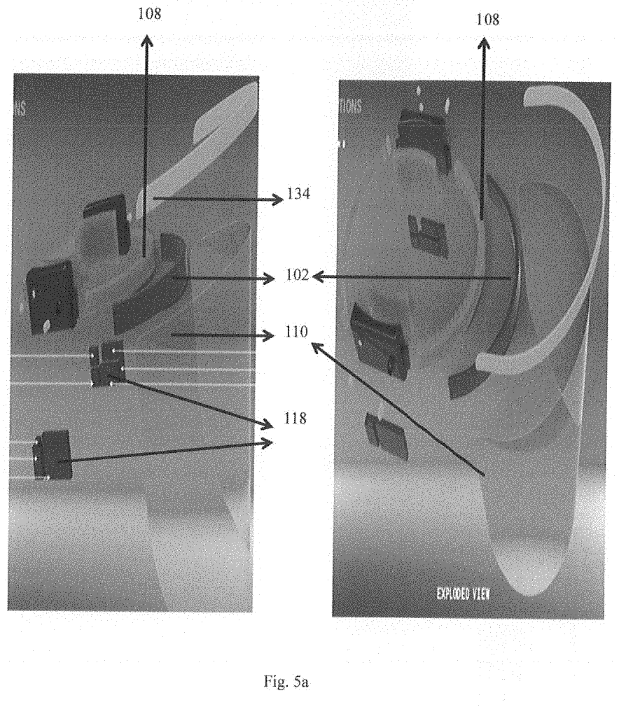

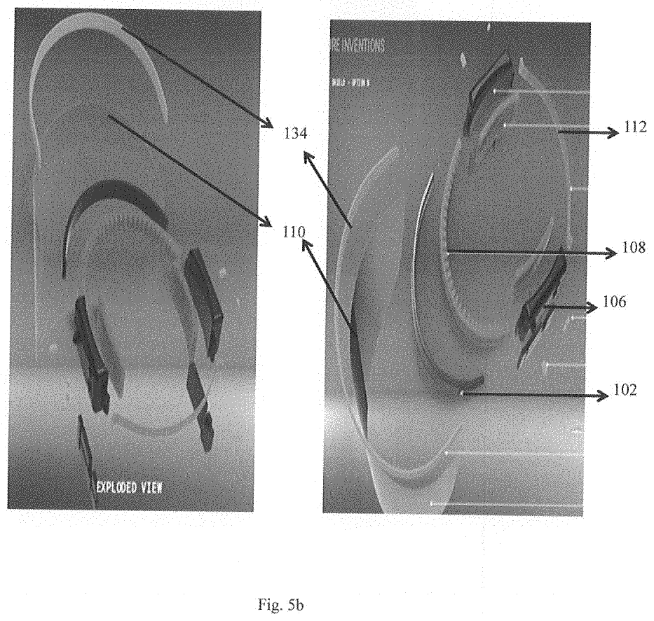

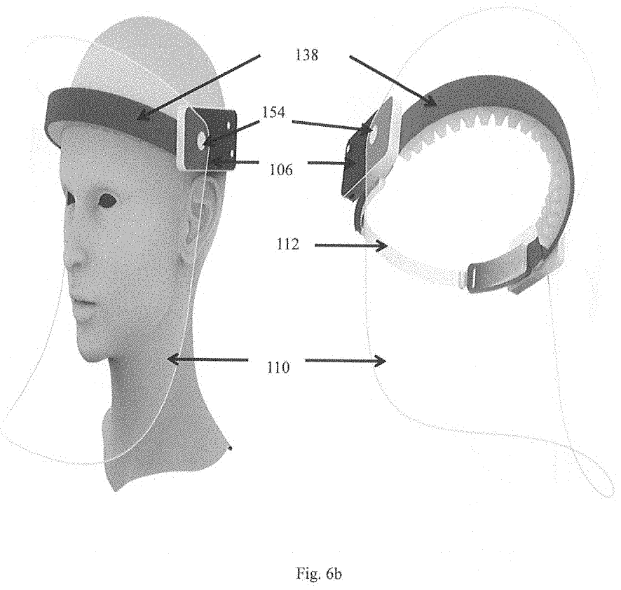

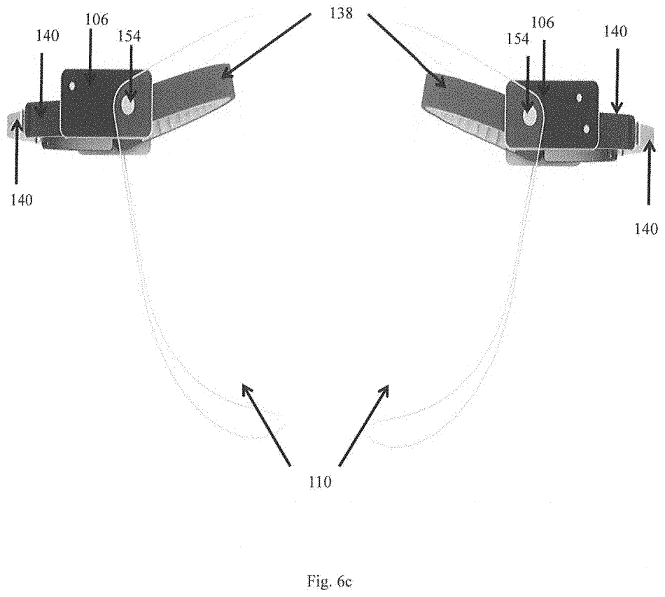

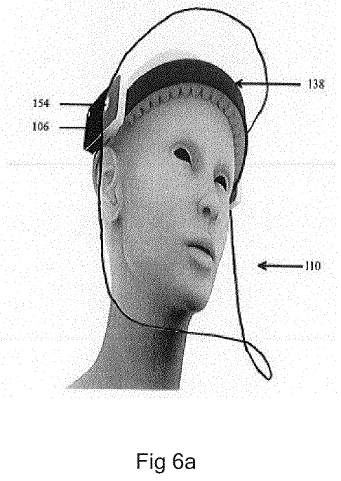

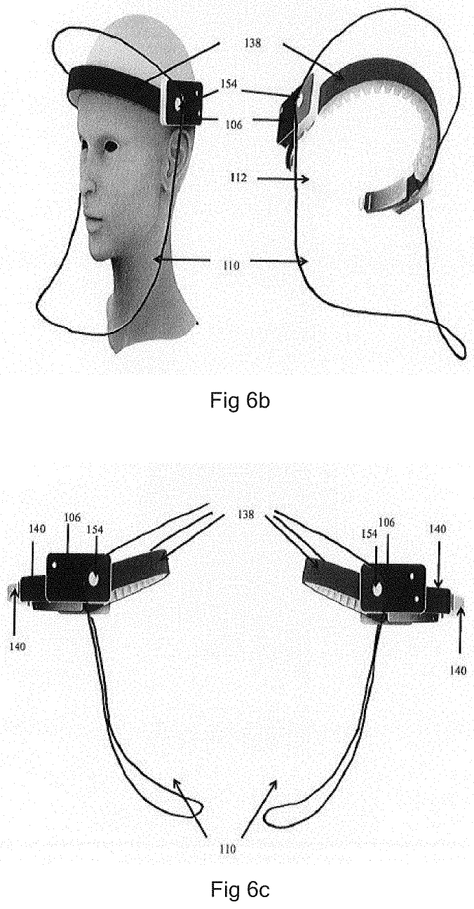

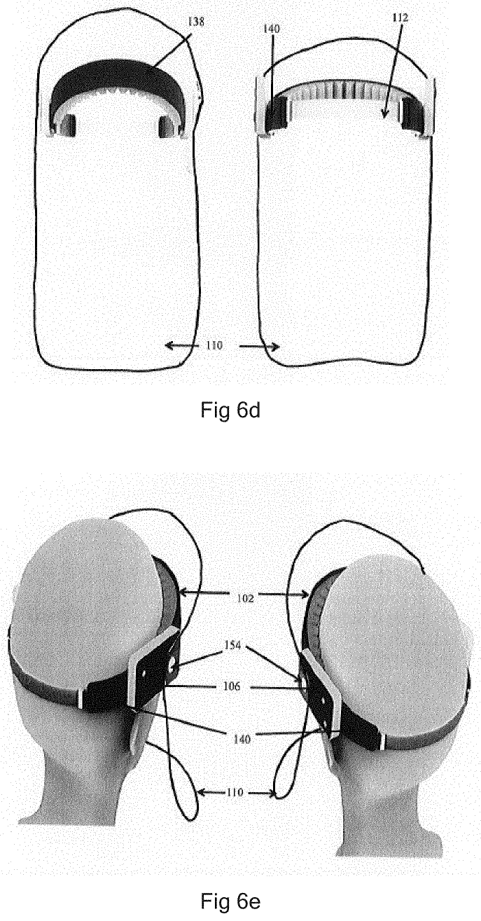

The rotatable face shield 110 is adapted to rotate against the frame 102. The shield 110 may comprise a lining 134, which may be made of silicone or other flexible material. The device 100 may comprise a connection point 136 at the outer surface 106o of each of the shell 106. The lining 134 may be connected to the device 100 at the connection point 136. In an embodiment, the connection point 136 may comprise a bolted joint. The connection point 136 may be configured to be rotated. As shown in FIGS. 6a to 6g, the rotations possible with the shield 110 are disclosed. The lining 134 may be evenly steep connected to the shell 106 to enable its rotational movement. In an embodiment, the lining 134 may be configured to be adjusted to at least three positions (P1, P2, P3) longitudinally along the face of the user. In another embodiment, the shield 110 may be configured to be adjusted to more than three or less than three positions (P1, P2, P3) along the face of the user. The angle between the shield at position P3 and a line of reference along the lining may be greater than the angel between the shield 110 at position P2 and the line of reference along the lining. Further, the angle between the shield at position P1 and the line of reference is less than the angle between the shield 110 at position P2 and the line of reference along the lining FIGS. 5a and 5b illustrate an exploded view of the portable face shield, in accordance with an embodiment of the present invention. The at least three positions (P1, P2, P3) of the shield 110 may offer flexibility to the device 100 and allows the user to adjust the shield 110 to any of the positions P1, P2, or P3 (and other positions) based on the comfort. In an embodiment, the device 100 may allow the user to move the head easily without letting the device 100 hurt the head or face of the user. For example, the user may be able to move his/her head up (towards the ceiling) and the shield may be adjusted accordingly. Both the ends of the frame 102 running through shell 106 are integrally connected to the fastening unit 112 as shown in FIG. 4a.

FIGS. 6a and 6b illustrate a perspective view of the device 100 in accordance with an embodiment of the present disclosure. The portable face shield 100 may comprise a frame 102 for resting the device 100 on the head of the user. The frame 102 may comprise a rotatable band 138 configured to be rotated along the head of the user, and at least one connecting unit 140 on each of the ends of the rotatable band 138. The portable face shield 100 may further comprise of a fastening unit 112, preferably an elastic band to provide a grip on the user's head to hold the portable face shield 100 from behind, a face shield 110 (hereinafter referred to as “shield”) detachably connected to the frame 102 by one or more shield locking buttons 154 and a pad 108. Further, the device 100 may comprise at least one shell 106 coupled to the frame 102 and adapted for housing an electronic circuitry 104.

According to an embodiment, the connection between the frame 100 and the at least one shell 106 through the connecting unit 140 may allow rotation of the frame 102. FIG. 6c illustrates a left and right side view of the device 100, in accordance with an embodiment of the present disclosure. FIGS. 6d and 6e illustrate a front and back side of the device 100 in accordance with an embodiment of the present disclosure.

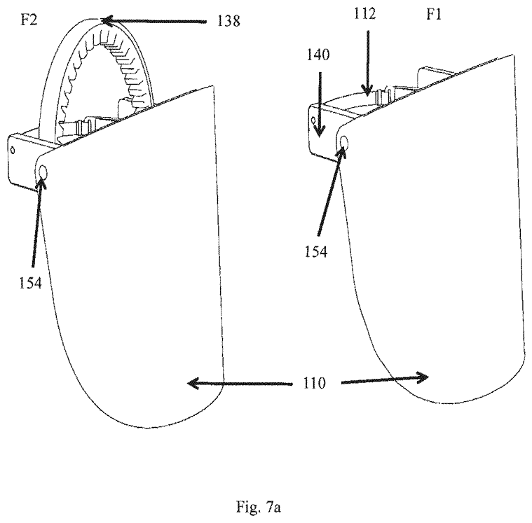

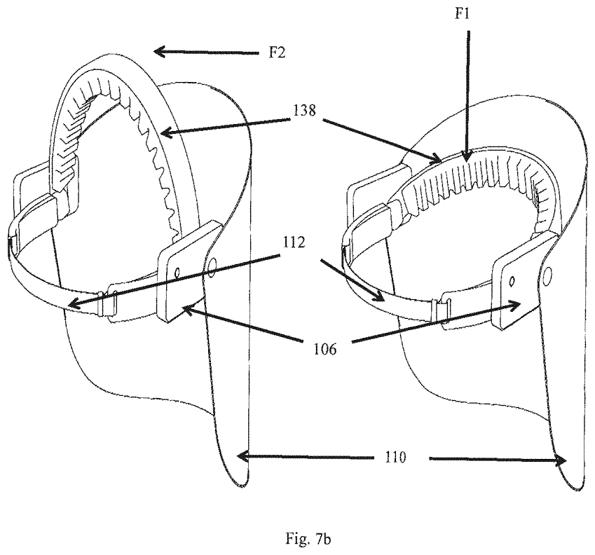

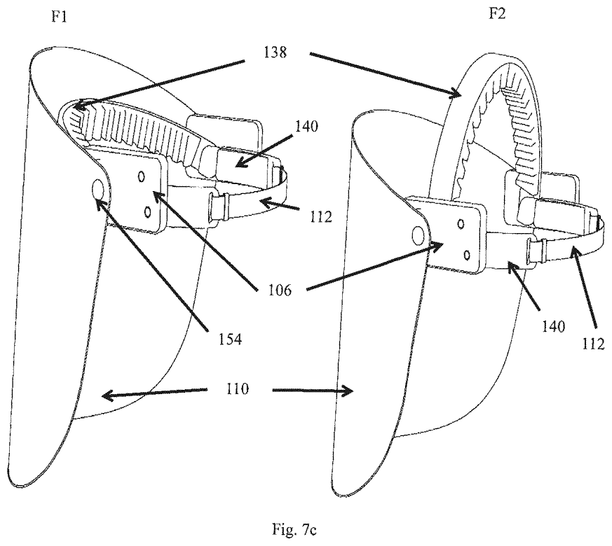

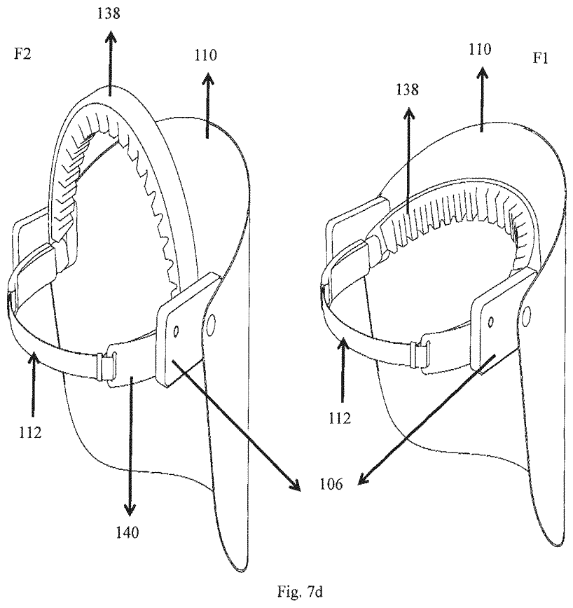

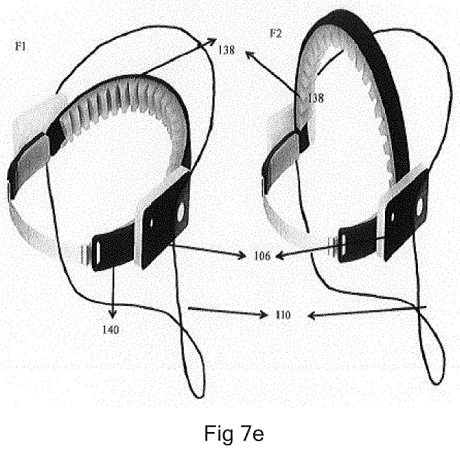

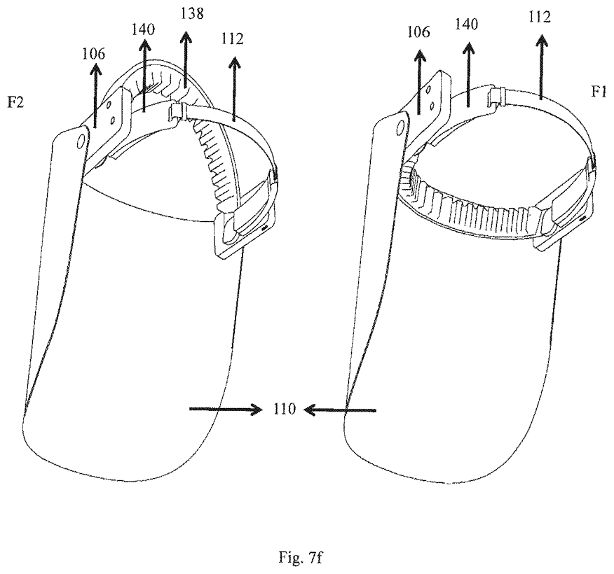

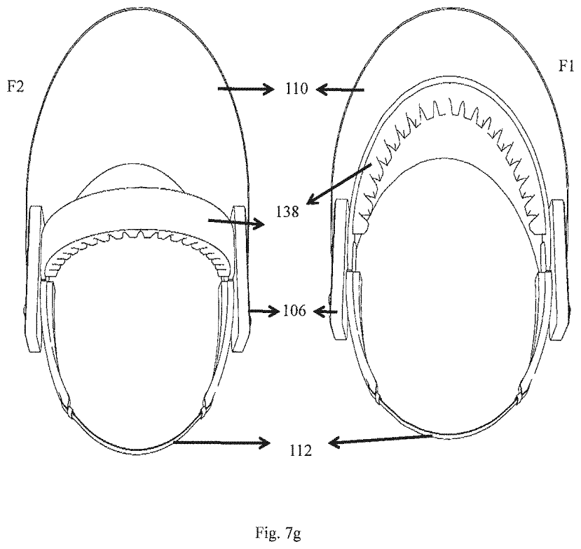

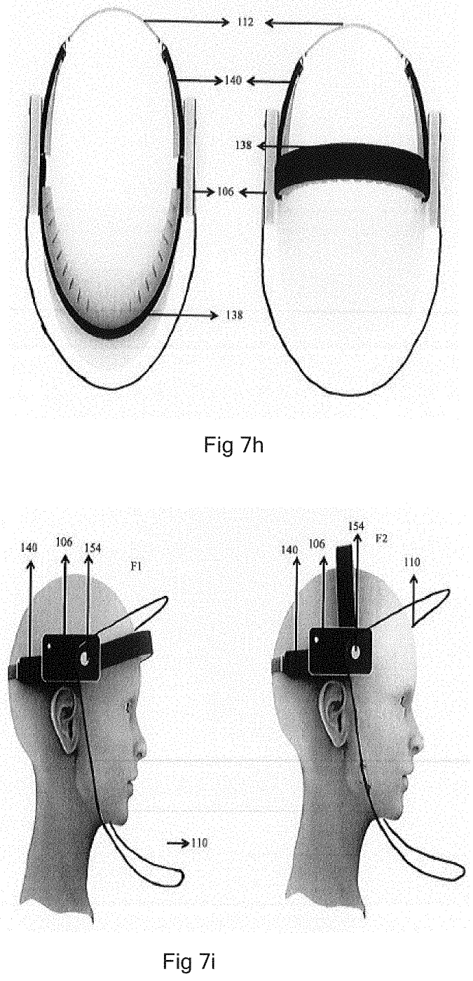

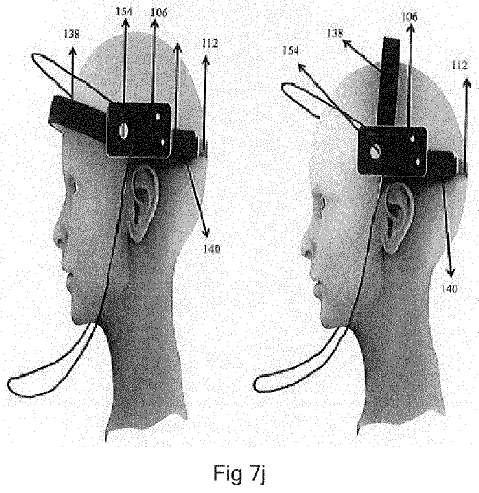

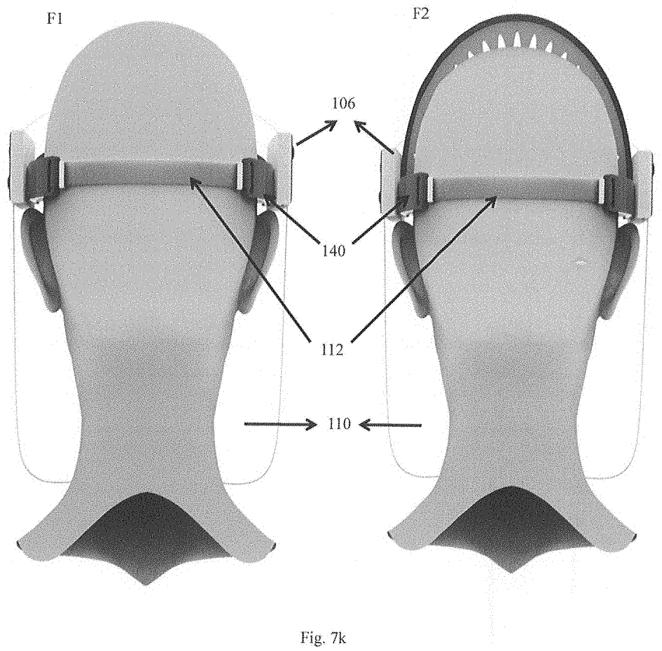

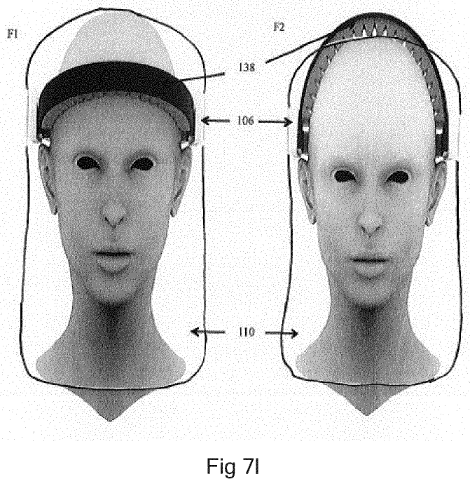

Further, referring to FIGS. 7a, 7b, 7c, 7d, 7e, 7f, 7g, 7h, 7i, 7j, 7k, and 7l illustrating different views of the device 100 in two different frame positions (herein after “positions (F1, F2)”), in accordance with an embodiment of the present disclosure. The rotatable band 138 may be configured to be positioned at at least two positions (F1, F2) along the head face of the user. In one of the positions the rotatable band 138 may be positioned on the forehead of the user, while in the other position, the rotatable band 138 may be positioned over the head of the user. Referring to FIG. 1, the inner surface 102i of the frame 102 may comprise a pad 108 fixed thereto. Therefore, the pad 108 may also rotate with the rotatable band 138 of the frame 102 and provide comfort to the user.

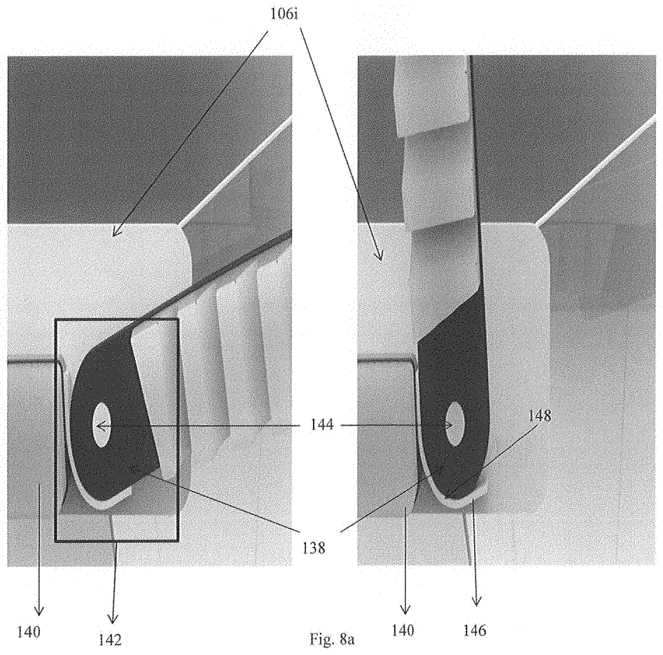

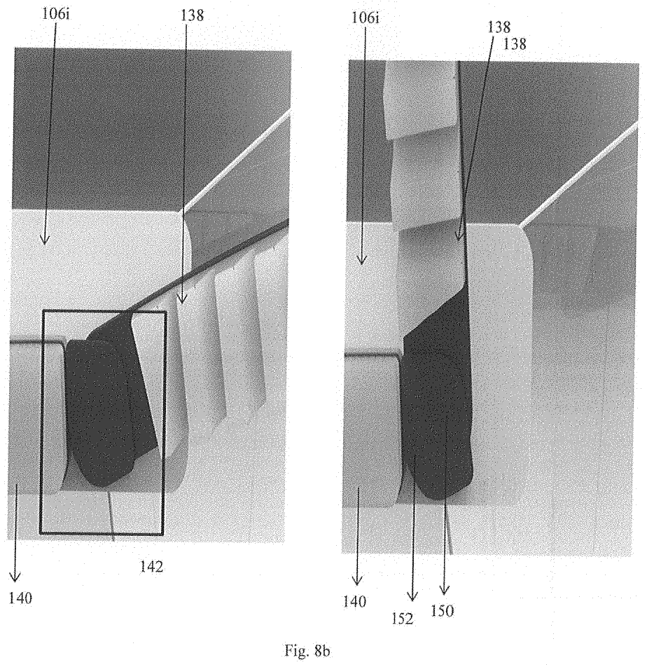

FIGS. 8a and 8b illustrates close up views of the connection points in the device 100 in two different rotatable band positions (F1, F2), in accordance with an embodiment of the present disclosure. Referring to FIG. 8a, the rotatable band 138 may be connected to the inner surface 106i of the shell 106 that comes in contact with the head of the user wearing the device 100. The surface 106i of the shell 106 may comprise a locking mechanism 142 for locking the movement of the rotatable band 138 at certain positions. The locking mechanism 142 may comprise a protruding boundary 146 on the surface 106i of the shell 106 and around a joint 144 of the rotatable band 138 and the shell 106. The protruding boundary 146 may from a parabolic curve 148 on the surface 106i of shell 106 such that the rotatable band 138 connected to the shell 106 at the joint 144 may be configured to be rotated along the boundaries of the curve 148 and may be locked at the ends of the curve 148. In an embodiment, one end of the curve 148 may be adjacent to end of the connecting unit 140 fixed to the inner surface 106i of the shell 106. Therefore, the locking mechanism may prevent the rotatable band 138 from rotating rotate beyond the boundaries of the curve 148 and also prevent it from slippage.

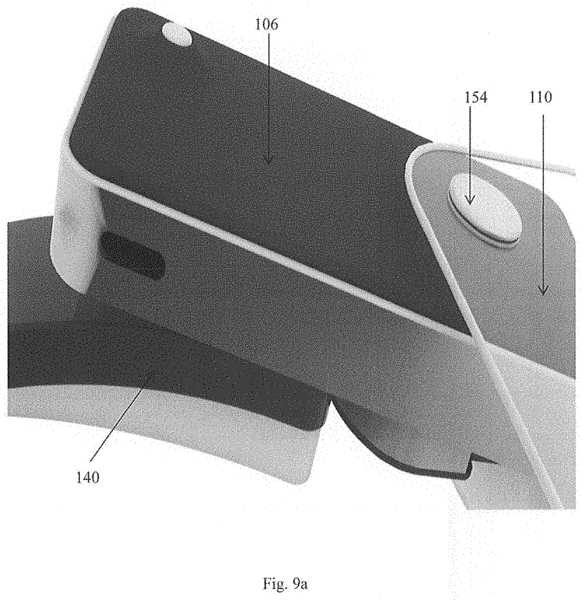

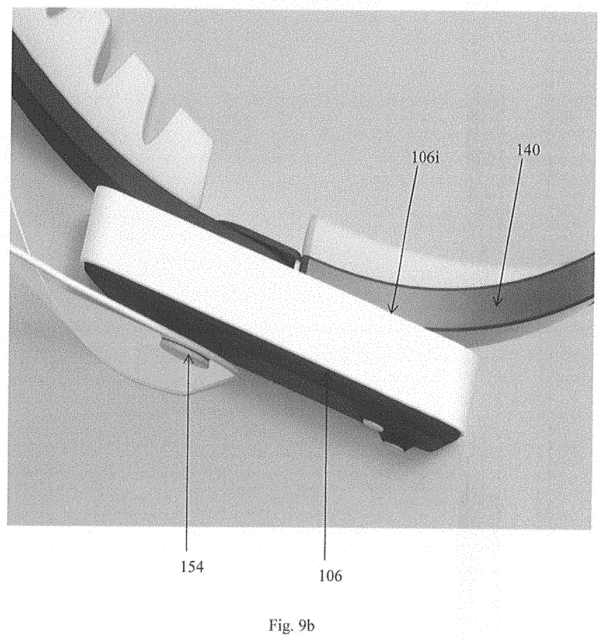

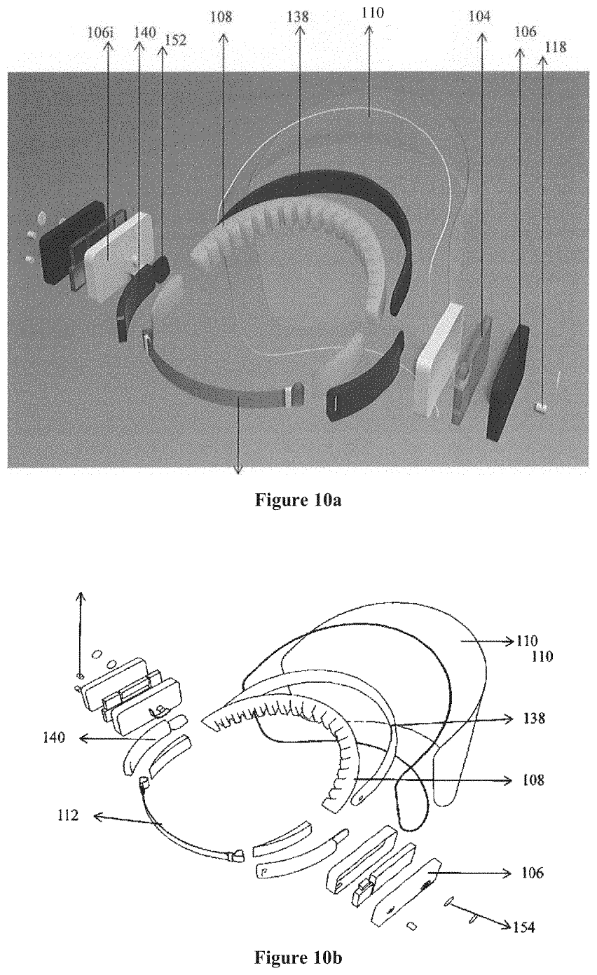

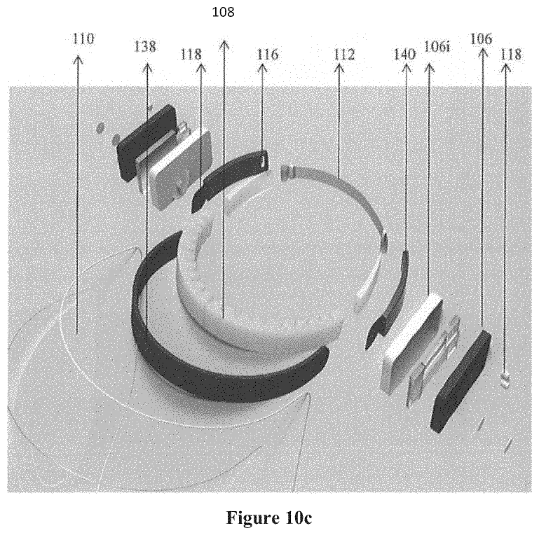

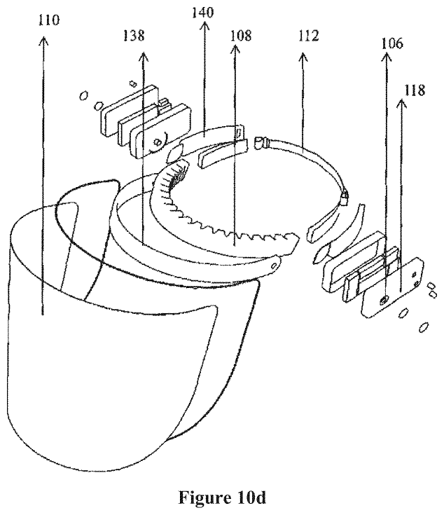

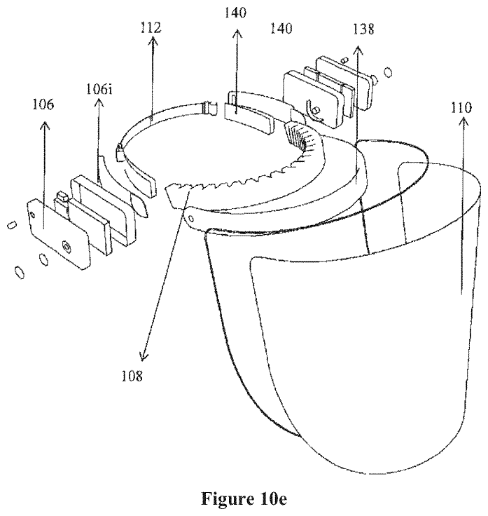

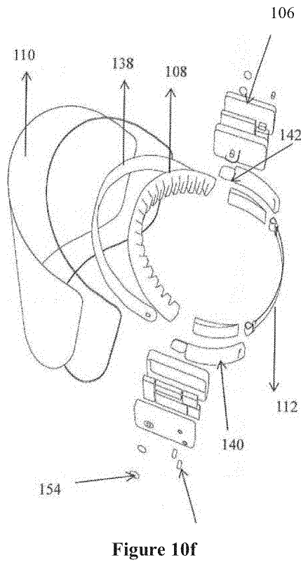

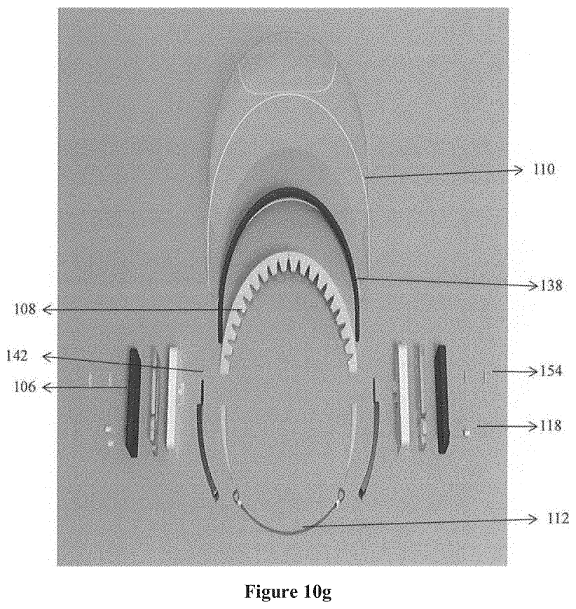

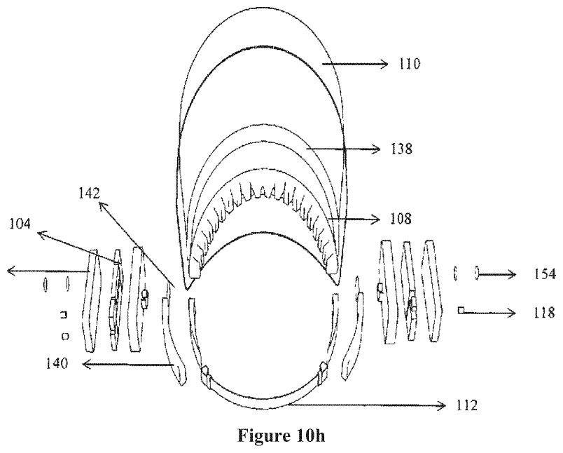

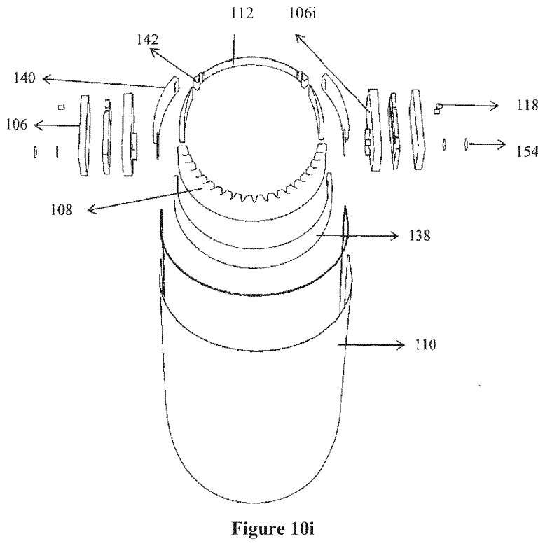

In another embodiment, referring to FIG. 8b, the locking mechanism 142 may comprise a sliding lock 150. The sliding lock 150 may be made by connecting the end of the rotatable band 138 to an extended surface 152 of the one of the ends of the connecting unit 140 fixed to the inner surface 106i of the shell 106. In an embodiment, the extended surface 152 may be a leaf like structure made of plastic or like material. The extended surface 152 and the end of the rotatable band 138 may be connected in an overlapping manner with preferably the extended surface 152 of the end of the rotatable band 138 such that the band may freely rotate along the joint 144. The rotation of the rotatable band 138 may be prevented by the ends of the connecting unit 140 that are fixed to the shell 106 at inner surface 106i. FIGS. 9a and 9b illustrate the close up outer and top view of the device at the placement of the shield 106. FIGS. 10a-10i illustrate different exploded views of the device 100, in accordance with an embodiment of the present disclosure.

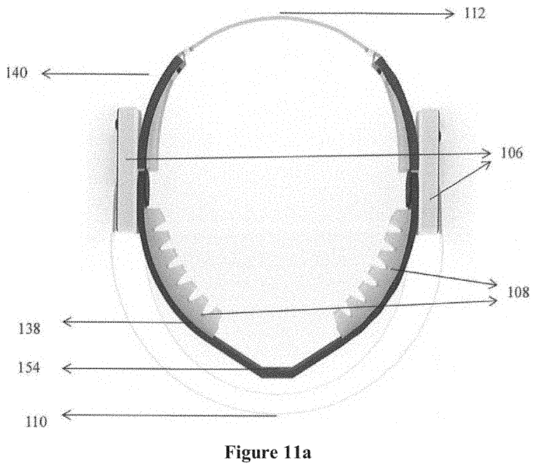

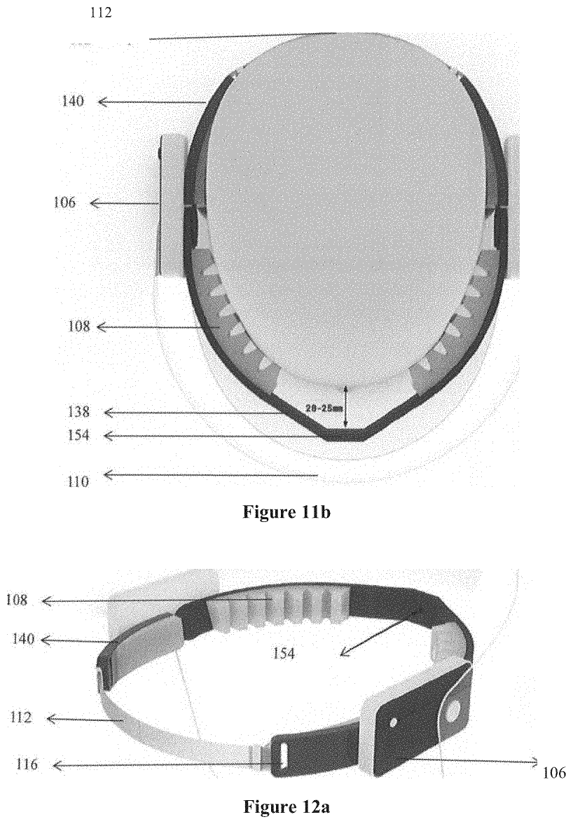

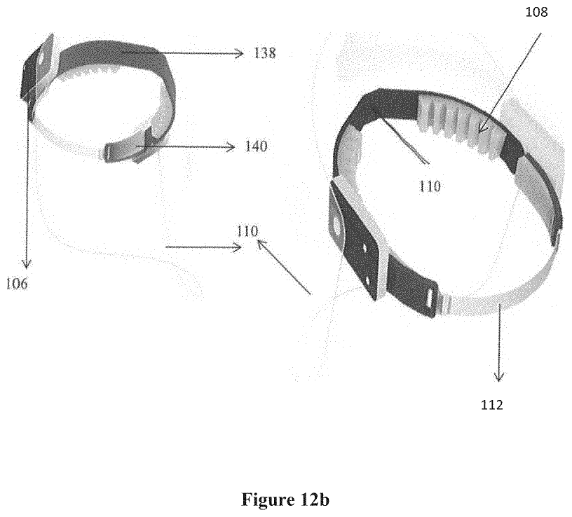

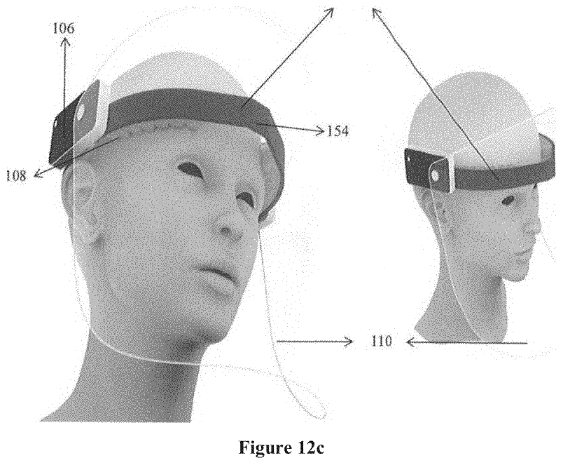

FIGS. 11a and 11b illustrate an overall schematic view of a portable face shield 100 in accordance with an embodiment of the present disclosure. The portable face shield device 100 may comprise a heat sensor 154 positioned at the centre of the frame 102, according to an embodiment. According to another embodiment, the heat sensor may be positioned at the centre of the inner surface of the rotatable band 138, if the face shield device 100 has rotatable configuration as illustrated in FIGS. 7a-7l. According to an embodiment, as illustrated in FIG. 12b, the face shield device 100 may comprise the pad 108 divided into two portions of equal length and having a hollow portion between the two portions. The two equal portions of the pad 108 may be positioned on the head of the user at two sides. The heat sensor 154 may be positioned at the centre between two equal portions. According to an embodiment, the heat sensor 154 may be positioned at a distance ranging between 20-25 mm from the head of the user wearing the device 100 (FIG. 12b). FIGS. 12a-12c illustrate perspective view of the face shield device 100 comprising heat sensor 154.

In an embodiment, the heat sensor 154 may be configured to sense the temperature of the user wearing the face shield device 100. The heat sensor 154 may be a infrared sensor according to an embodiment. The heat sensor 154 may be connected to a processor configured to receive the temperature of the user detected by the heat sensor 154 and compare it with the pre-stored threshold temperature range. According to an embodiment, the threshold may be around 38.5 degrees Celsius. The processor may be connected to an indicator unit positioned at the outer surface of the frame 102. The indicator unit may indicate the user on the band, if the temperature sensed by the heat sensor is more than the threshold temperature. Therefore, the user and the people around the user may be indicated about the higher temperature of the user and may take adequate precautionary measures to prevent the spread of infection.

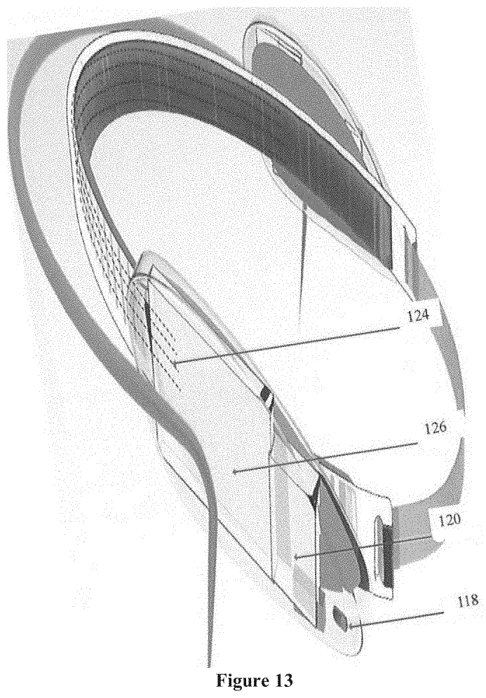

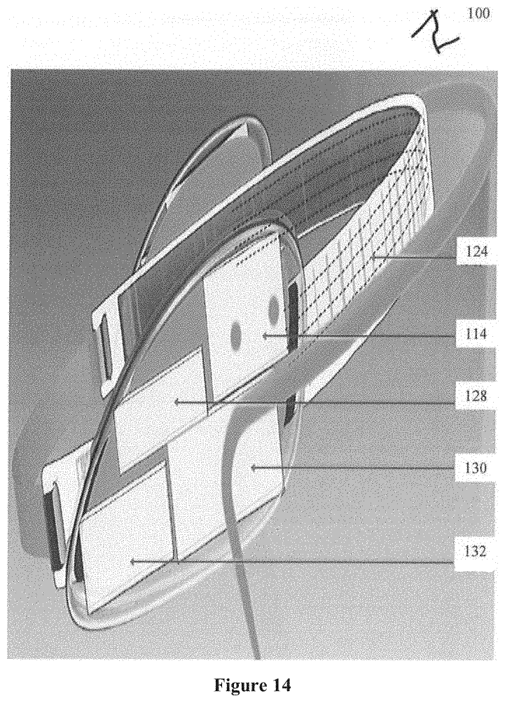

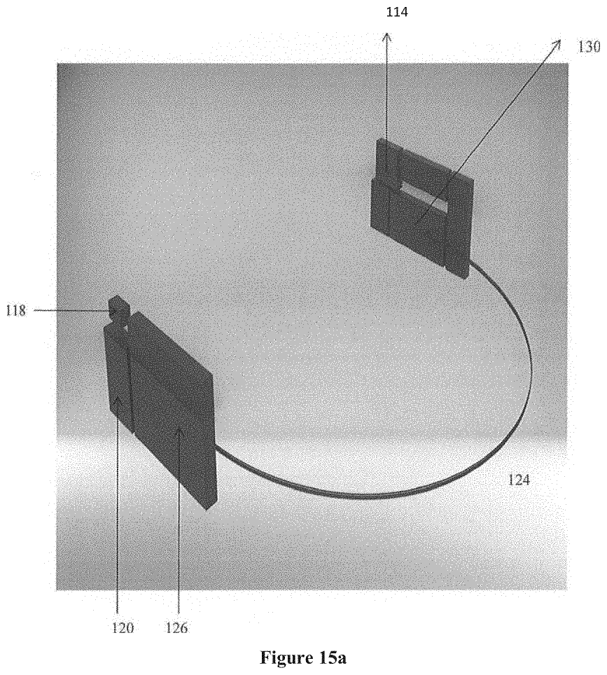

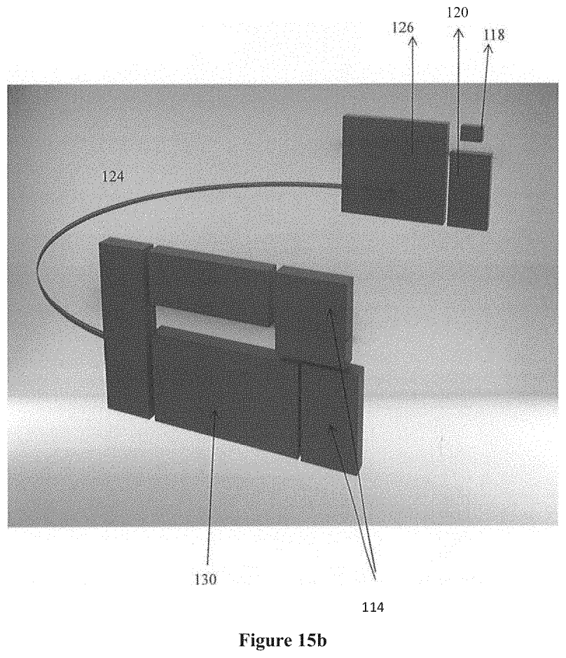

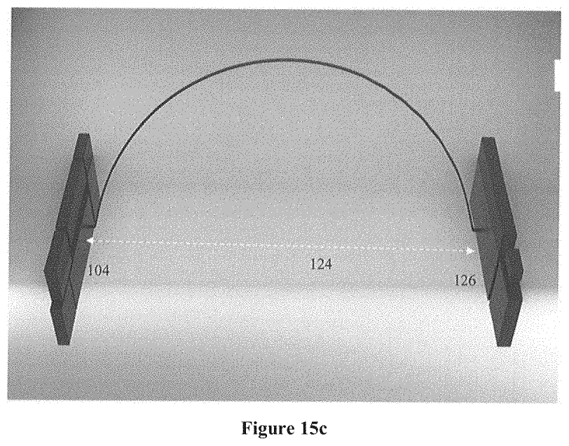

FIG. 13 and FIG. 14 illustrate respective left and right side X-ray view of electronic circuit components placement in a portable face shield, in accordance with an embodiment of the present disclosure. FIGS. 15a-15c illustrate different views of electronic circuit components placement in a portable face shield, in accordance with an embodiment of the present disclosure. The electronic circuit components may include but not limited to wires 124, battery 126, USB port 120, and switching button 118 according to an embodiment. In an embodiment, the buttons 118 may be made of silicone rubber material.

In an embodiment, the wires 124 may be connecting wires made of copper, enameled copper, PVC or like material. The wires 124 may run across the front portion of the device 100 between the frame 102 and the pad 104. The wires 124 may be connected to the battery 126 placed in the shell 106. In an embodiment, the battery 126 may be a rechargeable battery and/or replaceable battery. The battery 126 may be connected to the USB port 120. According to an embodiment, said shell 106 may comprise a microprocessor 130 in connection with one or more electronic components. In an embodiment, the microprocessor 130 may be an arduinonano. Further, the microprocessor 130 may be connected to a transceiver module 132. In an embodiment, the transceiver module 132 may be NRF24101 that may send and receive the data provided by the microprocessor 130.

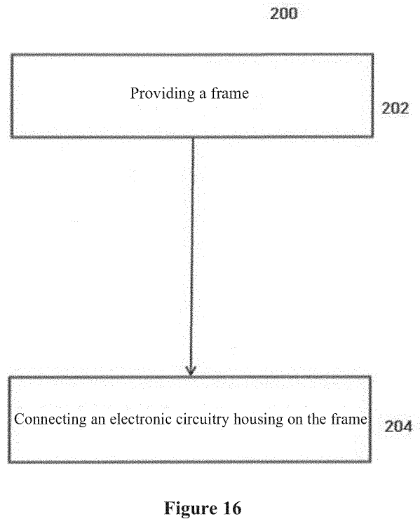

FIG. 16 illustrates a flow diagram depicting a process 200 of manufacturing a device 100, in accordance with an embodiment of the present disclosure. At step 202, the process 200 may comprise providing a frame 102 to be worn around the head of a user such that the frame 102 may be bendable and flexibly mountable around the head of the user. Further, at step 204, the process 200 may comprise connecting 206 the at least one shell 106 on the frame 102 such that the shell 106 house the electronic circuitry 104. Further, at step 206, the process 200 comprises fixing 204 an electronic circuitry 104 to the frame 102 at the outer surface 102i. Referring to FIGS. 4a and 4b, electronic circuitry 104 may comprise a plurality of electronic circuit components. In one embodiment of the present invention, the above mentioned described processed may be used to manufacture the portable face shield 100 and the portable face shield 100 with a rotatable face shield 100 as described in the embodiments above.

In an embodiment, the frame 102 may be bent and laser cut from bendable sheet material to form a semi-circular shaped configuration. The process may further comprise fixing the pad 108 to the inner surface 102i of the frame 102 such that wires 124 may be allowed to run between the frame 102 and the pad 108 according to an embodiment. In an embodiment, a further step may comprise connecting a fastening unit 112 to the two ends of the frame 102 at the slots 116 located at each end of the frame 102.

The portable face shield device 100 may prevent the virus from the surrounding from contacting the user and also may prevent the virus from spreading from the user to the surrounding. The portable face shield device 100 is light weight and is comfortable to wear for prolonged hours and also easy to remove off the head of the user. The portable face shield 100 is also appealing to the eyes of the user, and is particularly attractive to children, which prompts them to wear it and develops safety habits in them.

The smart face shield device 100 is enabled with adjustment in size based on the requirement (head) of the user and is stable even in the circumstances of sudden movement of the user. The portable face shield device 100 offers easy dismantling of the parts comprised therein, thereby allowing replacing the worn out parts with the new one without replacing the whole face shield. The worn out parts may include but not limited to the shield 110 and electronic circuit components. The portable face shield device 100 is, therefore, economic, easy to maintain, and has a longer life. Further, the portable face shield may allow tracking parameters such as acceleration, frequency, intensity, patterns in the movements, location, and directions related to user. It also facilitates compilation of the measured parameters and provide compiled results. The compiled results may be stored by the user in the host system by connecting it to the portable face shield by USB port 120. The measured parameters may also be compared with the preset values and indication may be provided to the user regarding the planning of further movement by the user.

The figures and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of the embodiments is by no means limited by these specific examples. Numerous variations, whether explicitly given in the specification or not, such as differences in structure, dimension, and use of material, are possible.

1. A face shield device (100) comprising:
a frame (102) for supporting the face shield device (100) on user's head; and
at least one shell (106) coupled to the frame (102) for housing an electronic circuitry (104).

2. The face shield device (100) of claim 1 further comprising at least one electronic circuitry comprising a plurality of electronic components for the operation of the device (100).

3. The face shield device (100) of claim 1, wherein the frame (102) comprises a belt and is U-shaped or semi-circular in shape.

4. The face shield device (100) of claim 3, wherein the frame (102) is symmetric about a centre axis and a variable such as thickness is constant around the circumference.

5. The face shield device (100) of claim 3, wherein the frame (102) is made of silicone, and is fixed to a pad (108).

6. The face shield device (100) of claim 5, wherein the pad (108) is made of polifoam.

7. The face shield device (100) of claim 1, wherein the at least one shell (106) are two shells (106), wherein the two shells (106) are positioned opposite each other on each side of the frame (102) such that the two shells (106) are symmetrical with respect to the center axis of the frame (102).

8. The face shield device (100) of claim 7, wherein a weight of the shell (106) is divided equally between the two shells (106) such that the weight of the circuitry (104) is distributed equally on both sides of the face shield device (100).

9. The face shield device (100) of claim 1, wherein the at least one shell (106) is made of polyethylene terephthalate glycol (PETG), and comprises a user interface for enabling the user to send a wireless signal.

10. The face shield device (100) of claim 9, wherein the user interface comprises a button (118), and the button (118) is a switching button.

11. The face shield device (100) of claim 9, wherein the at least one shell (106) is located above the ear of the user for enabling the user to access the user interface while wearing the device (100).

12. The face shield device (100) of claim 1 further comprising a shield (110) connected to the frame (102), wherein the shield (110) is made of polyethylene terephthalate glycol (PETG) or other transparent material and is removably connected to the frame (102).

13. The face shield device (100) of claim 12, wherein the shield (110) comprises a lining (134) on one of the sides adapted to be connected to the device (100), wherein the lining (134) is connected to the device (100) at one or more connection points (136) on the outer surface (106) of the shell (106).

14. The face shield device (100) of claim 13, wherein the one or more connection points (136) are bolted joint configured to be rotated against the frame (102), and wherein the one or more rotatable connection points (136) are configured to adjust the lining (134) of the shield (110) to at least three positions (P1, P2, P3) longitudinally along the face of the user.

15. The face shield device of claim 14, wherein the frame (102) comprises:
a rotatable band (138) configured to be rotated along the head of the user; and
a locking mechanism (142) for controlling the rotation of the rotatable band (138), wherein the locking mechanism (142) comprises a curve (148) on the inner surface (106i) of the shell (106) allowing the rotating of the rotatable band (138) along the boundaries of the curve (148) and wherein the locking mechanism (142) is configured to adjust the rotatable band (138) to at least two positions (F1, F2), wherein at one (F1) of the at least two positions (F1, F2), the rotatable band (138) is on the forehead of the user.

16. The face shield device (100) of claim 14, further comprising a heat sensor (154) positioned at the center of the frame (102) of the device (100) to sense the body temperature of the user, wherein heat sensor (154) is connected to a processor, the processor being configured to receive a temperature of the user detected by the heat sensor (154) and to compare the detected temperature with a pre-stored threshold temperature range.

17. The face shield device (100) of claim 16, wherein the processor is connected to an indicator unit, wherein the indicator unit indicates the user when the detected temperature of the user is greater than the pre-stored threshold temperature range.

18. A process (200) of manufacturing a face shield device (100) comprising a frame (102) and at least one shell (106) coupled to the frame (102) for housing the electronic circuitry, the process (200) comprising:
providing the frame (202) to be worn around the head of a user; and
connecting (204) the at least one shell (106) on the frame such that the circuitry housing house the electronic circuitry (102).

19. The process (200) of claim 18, further comprising fixing (206) the electronic circuitry to the frame (102), wherein the frame (102) is bent and laser cut from a bendable sheet material to form a semi-circular shaped configuration.

20. The process (200) of claim 19, further comprising fixing a pad (108) to the frame (102).
